(19) United States
(12) Patent Application Publication
(10) Pub. No.: US 2019/0144574 A1
(43) Pub. Date: May 16, 2019

(54) METHOD FOR PREPARING AN AQUEOUS POLYACRYLAMIDE SOLUTION

(71) Applicant: BASF SE, Ludwigshafen am Rhein (DE)

(72) Inventors: Hazel SPRAFKE, Lampertheim (DE); Faissal-Ali EL-TOUFAILI, Ludwigshafen (DE); Bjoern LANGLOTZ, Ludwigshafen (DE); John BARRATT, Bradford (GB); Tobias BRINGMANN, Ludwigshafen (DE); Gabriela Eugenia FONSECA ZEPEDA, Ludwigshafen (DE); Sandra JECK, Ludwigshafen (DE); Dennis LOESCH, Ludwigshafen (DE); Dirk MECKELNBURG, Ludwigshafen (DE); Markus OSTERMAYR, Ludwigshafen (DE); Bernd SCHUBE, Ludwigshafen (DE); Oliver SOETJE, De Meern (NL)

(73) Assignee: BASF SE, Ludwigshafen am Rhein (DE)

(21) Appl. No.: 16/096,169

(22) PCT Filed: Apr. 25, 2017
(86) PCT No.: PCT/EP2017/059759

§ 371 Date: Oct. 24, 2018

(30) Foreign Application Priority Data
Apr. 26, 2016 (EP) 16167080.7

(51) Int. Cl. C08F 20/56; C08F 2/10; C08F 2/01; C08F 6/06

(52) U.S. Cl. CPC C08F 20/56; C08F 2/10; C12P 13/02; C08F 6/06; C08F 2/01

(57) ABSTRACT

A method for preparing an aqueous polyacrylamide solution is disclosed. The method comprises:—hydrating acrylonitrile in water in presence of a biocatalyst capable of converting acrylonitrile to acrylamide so as to obtain an acrylamide solution,—directly polymerizing the acrylamide solution so as to obtain a polyacrylamide gel, and—directly dissolving the polyacrylamide gel by addition of water so as to obtain an aqueous polyacrylamide solution.

### FIELD OF THE INVENTION

The present invention relates to a method for preparing an aqueous polyacrylamide solution.

### RELATED ART

Polyacrylamides and their copolymers with other monomers are utilized in many applications such as mining, water treatment, sewage treatment, papermaking, oil well drilling, oil production, and agriculture. Common co-monomers for acrylamide are acrylic acid and its salts (“anionic polyacrylamide”) as well as cationic ester of acrylic acid (“cationic acrylamide”). The utility of these polymers is directly related to their chemical structure, functionality, and molecular mass. The high polymerizability of the monomers allows the preparation of high molecular mass polymers, which are useful as flocculants and thickeners.

High molecular weight polyacrylamides having a weight average molecular weight of more >10⁶ g/mol may be used in the exploration and production of mineral oil, in particular as rheology modifier for aqueous drilling fluids or as thickeners in aqueous injection fluids for enhanced oil recovery. Enhanced oil recovery techniques using polymer thickened aqueous fluids are also known as “polymer flooding”. Furthermore, high molecular weight polyacrylamides may also be used as flocculating agent for tailings and slurries in mining activities.

Such high molecular weight polyacrylamides may in particular be made by gel polymerization. In gel polymerization an aqueous monomer solution having a relatively high concentration of monomers, for example from 20% by weight to 35% by weight is polymerized by means of suitable polymerization initiators thereby forming a solid polymer gel. The polymer gels formed are converted to polymer powders by comminuting the gel into smaller pieces by one or more size reduction steps, drying such gel pieces for example in a fluid bed dryer followed by sieving, grinding and packaging. Lubricants and anti-sticking aids are usually used to facilitate the processing of the polymer gel. The obtained powders are packaged and shipped to customers.

For use in polymer flooding or mining applications dilute aqueous solutions of polyacrylamides are used. Typical concentrations of the polymer range from 0.05 wt. % to 0.5 wt. %. Consequently, for use the powders of polyacrylamides have to be dissolved again in aqueous fluids. Dissolving high molecular weight polymers in water is time consuming and it is difficult to do so without degrading the polymers. It is necessary for the customers to have available on-site suitable equipment for dissolving said high molecular weight powders of polyacrylamides.

The polymer gel obtained from gel polymerization typically comprises from 65% to 80% of water. The abovementioned powders of polyacrylamides still comprise some residual water which may be from 4 to 12% by weight. So, drying the polymer gels does not mean to remove some residual moisture but per kg of polymer gel about 0.55 to 0.75 kg of water need to be removed, or—with other words—per kg of polymer powder produced also 1.5 to 2.5 kg of water are also “produced”.

It goes without saying that drying such gels is energy extensive and consequently the operational costs for drying are high. It also goes without saying that high-performance dryers are necessary in order to dry the polymer gels. Furthermore, also equipment for the other post-processing steps size reduction, sieving and grinding is necessary. Consequently, the capital expenditure for the entire post-processing, size reduction, drying, sieving, grinding is significant in relation to the total capital expenditure. Furthermore, the process steps after cutting the wet polymer gel typically involve a lot of dust creating processing steps such as fluid bed drying, grinding, milling, pneumatic transport, packing, transport to customer location, unpacking, dosing into dissolution equipment and the like. This polymer dust is either scrapped or with high effort it is targeted to keep the dust in the process by incorporating it in the final product. However, dust emissions to the ambient still occur e.g. at the unloading or final dissolution step of the customer. All the above mentioned points represent either product losses, exposure to workers or waste of energy.

For enhanced oil recovery or for mining applications large amounts of polyacrylamides need to be available at one location, i.e. at an oilfield or at a mining area. For example, even for flooding only a medium size oilfield it may be necessary to inject some thousand m³ of polymer solution per day into the oil bearing formation and usually the process of polymer flooding continues for months or even years. For a polymer concentration of only 0.2 wt. % and an injection rate of 5000 m³ 10 t of polymer powder are needed per day and need to be dissolved in an aqueous fluid.

It has been suggested to manufacture polyacrylamides on-site.

ZA 8303812 discloses a process for preparing polyacrylamides comprising polymerizing acryl amide and optionally suitable comonomers on-site and transferring the polymer formed to its desired place of use on site without drying or concentrating. The polymerization can be carried out as an emulsion polymerization, bead polymerization, or as solution/dispersion polymerization. The polymer may be pumped from the polymerization reactor to the position on site where it is used.

U.S. Pat. No. 4,605,689 A describes a 2-step process for converting polyacrylamide gel, preferably comprising from 6 to 15% by weight of solid polymer into dilute aqueous solutions suitable for use in secondary oil recovery. Polyacrylamide gel is initially converted into a slurry of small gel particles in water which forms a homogeneous solution concentrate which is then readily diluted to give the final drive fluid without any significant polymer degradation. The gel solution is passed through static cutting units with available water in order to provide a uniform slurry of particulate gel solids having a desired polymer solids content without substantially degrading the polymer, i.e., reducing its molecular weight.

WO 2016/006556 A1 describes a method for producing a compound using a continuous tank reactor which is provided with two or more reaction tanks for producing the compound and with a reaction liquid feeding pipe that feeds a reaction liquid from an upstream reaction tank to a downstream reaction tank, said method being characterized in that the Reynold's number of the reaction liquid that flows in the reaction liquid feeding pipe is configured to be 1800-22000. The compound may be acrylamide produced by conversion from acrylonitrile by means of a biocatalyst. The tank reactor may be mounted in a portable container. However, WO 2016/006556 A1 does not disclose any further processing of the acryl amide solution obtained.

Despite said suggestions, most of the polyacrylamides for use in mining and oilfield applications are sold nowadays as powder, although this requires also cost intensive setup and a lot of know how to be re-dissolved on site of application.

One of the reasons for the failure are the transport costs of the aqueous acryl amide solution to remote locations. Acryl amide typically is manufactured by hydrolysis of acrylonitrile in the presence of a suitable catalyst. It is known in the art to use a copper catalyst such as Raney copper for hydrolysis. The hydrolysis is performed at temperatures of about 120° C. under pressure. The catalyst is separated from the reaction mixture and recycled and also non-hydrolyzed acrylonitrile has to be recycled. The process yields an aqueous solution comprising about 30 to 50% by wt. of acrylamide. It is also known in the art to use biocatalysts such as nitrile hydratase. With biocatalysts hydrolysis is already possible at low temperatures and low pressures. The process also yields an aqueous solution comprising about 30 to 50% by wt. of acrylamide. So, using a 30 to 50% aqueous solution of acryl amide means to transport at least double as much material compared to transporting only polyacrylamide powder.

An object of the present invention is to provide a process for preparing an aqueous polyacrylamide solution that is suitable to minimize or overcome the above problems. Particularly, it is an object of the present invention to provide a process for preparing an aqueous polyacrylamide solution that allows energy saving, compact and transportable installation for on-site production of polyacrylamide or copolymers of acrylamide.

### SUMMARY

Disclosed herein is a method for preparing an aqueous polyacrylamide solution.

Embodiments of the disclosed method have the features of the independent claim. Particular embodiments, which might be realized in an isolated fashion or in any arbitrary combination, are listed in the dependent claims.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “preferably”, “more preferably” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

A method for preparing an aqueous polyacrylamide solution according to the present invention comprises the following steps, particularly in the given order:
hydrating acrylonitrile in water in presence of a biocatalyst capable of converting acrylonitrile to acrylamide so as to obtain an acrylamide solution,
directly polymerizing the acrylamide solution so as to obtain a polyacrylamide gel, and
directly dissolving the polyacrylamide gel by addition of water so as to obtain an aqueous polyacrylamide solution.

The term “directly” as used herein is to be understood that two steps of the method according to the present invention are carried out immediately in a subsequent order such that there is a continuous process of these two steps. This directly processing excludes any unnecessary or technically unavoidable delay between two subsequent process steps. Therefore, these two process steps may be interrupted only by unexpected or technically unavoidable events in order to be directly carried out in the sense as used herein. Thus, a product resulting from a previous method step is not stored for a certain time, transported by external devices such as ships or vehicles and supplied to a site for carrying out the subsequent process step but there is a direct connection between the two method steps. With other words, the term “directly” is to be understood as “by means of a direct connection”. Needless to say, this does not exclude any process steps that are carried out in-line such as a removal or separation of certain ingredients by means of filtration or the supply of any additives such as water. Needless to say, if technical applications require so, the product from a previous method step may be temporarily buffered. For example, “directly polymerizing an acrylamide solution” means that the acrylamide solution resulting from converting acrylonitrile to acrylamide at a first site is not stored and/or transported to a second site but is directly supplied from the first site to the second site such as by means of pipes, lines or the like, wherein the pipes, lines or the like connect the first site to the second site via a buffer tank. Thus, the polymerizing process immediately starts with the end of converting acrylonitrile to acrylamide. Accordingly, a time gap between converting acrylonitrile to acrylamide and polymerizing the resulting acrylamide is decreased to a minimum.

The term “acrylamide” shall also include methacrylamide. Preferably, the term “acrylamide” shall mean acrylamide as such.

Hydrating acrylonitrile in water in presence of a biocatalyst capable of converting acrylonitrile to acrylamide so as to obtain an acrylamide solution avoids the use of any potential problematic catalysts such as copper which may in principle also used for converting acrylonitrile to acrylamide. Thus, the use of a biocatalyst avoids any waste problems. Further, by means of using biocatalysts for converting acrylonitrile to acrylamide instead of other catalysts such as copper, the acrylamide monomer can be easily produced at ambient pressure and temperature such that heating is voided which was otherwise necessary. This allows the production of the polymer on site starting with acrylonitrile. Thereby, energy may be saved and the conversion may be carried out at ambient temperature. The transport costs of acrylonitrile are even lower than that of the polymer as each kg of acrylonitrile makes about 1.5 kg of solid polymer. On volume basis the calculation are even much more preferable for acrylonitrile due to the low bulk density of the polymer powder.

For polymerization the aqueous acrylamide solution obtained in the first step may be used as such thereby obtaining homo polyacrylamide. Preferably, the aqueous solution may be mixed with one or more monoethylenically unsaturated, water-soluble comonomers thereby obtaining copolymers comprising acryl amide and one or more comonomers. Suitable monoethylenically unsaturated comonomers are mentioned below. In one embodiment of the invention, acrylic acid and/or 2-acrylamido-2-methylpropane sulfonic acid or salts thereof may be used as comonomer(s). As the aqueous solution comprising acryl amide is directly polymerized so as to obtain a polyacrylamide gel, significant costs for transport of aqueous solutions of acryl amide to remote locations may be saved.

The concentration of the monomers in the aqueous monomer solution shall be such that an aqueous polymer gel is formed upon polymerization. Such an aqueous gel may be regarded as a polymer-water system in which there is a three-dimensional network structure composed of macromolecules or their associates and which is capable of retaining significant amounts of water. The network is formed by physical forces. Such a system keeps its shape under the action of its own weight and differs in this feature from a polymer solution. Suitable definition of a polymer gel is given in the article by L. Z. Rogovina et al., Polymer Science, Ser. C, 2008, Vol. 50, No. 1, pp. 85-92.

The aqueous polyacrylamide polymer gel should comprise at least 10% by weight of polyacrylamides. The polyacrylamide gel may comprise 16% to 50% by weight, preferably 18% to 48%, more preferably 20% to 45% even more preferably 25% to 40% and still more preferably 32% to 38% polyacrylamide solids.

Directly dissolving the polyacrylamide gel by addition of water so as to obtain an aqueous polyacrylamide solution improves the product quality of the resulting aqueous polyacrylamide solution. Particularly, with conventional processes for preparing aqueous polyacrylamide solutions water-soluble polymers in the form of dry polymer powders are provided and made up into aqueous polymer solutions at the site where they are intended to be used. This typically involves dispersing the dry polymer powders into water and allowing the polymer powder to hydrate and gradually dissolve. This is normally achieved by employing make up equipment. Water-soluble particulate polymers are by nature hygroscopic and are notoriously difficult to add to water in order to mix into homogenous aqueous solutions. If the powder is added to water incorrectly, the hydrating polymer particles can stick to the make up equipment and/or to each other, resulting in lumps or agglomerates of polymer in the aqueous polymer solution. Unfortunately, such lumps or agglomerates tend not to dissolve once they have formed. It is normally important that the solutions of polymer are substantially homogenous, since otherwise in the various chemical treatment applications to which these solutions are applied, the dosing equipment may become blocked or lumps/agglomerates may adversely affect the particular process. Since water-soluble polymers readily absorb water and become sticky, care has to be taken in the transfer of dry polymer powder into the make up equipment. Desirably the particles of the polymer should remain as individual entities and hydrate separately. However, material wetting and make up equipment can become blocked because the particulate material becomes hydrated prematurely. This can happen if particles stick to damp services. Frequently, this can happen in the proximity of the wetting equipment where water is done by with the particulate material, for instance, where too much particulate material or agglomerates of material is fed into the mixing equipment. This often results in this part of the equipment becoming blocked with gel or with layers of concretions which can stop the process and/or cause spillage of particulate material. Consequently, the operation will require regular maintenance. Thus, as the preparation of powder is avoided with the method according to the present invention, not only significant costs for drying, grinding and the like of the polyacrylamide and the preparation of powder are saved, but the solubility and homogenization of the polyacrylamide is significantly better.

The dissolution of the aqueous polyacrylamide gel in water may be performed by any technique.

In an embodiment, the gel may simply be mixed with water in a suitable vessel while stirring. In order to ensure a rapid dissolution of the polyacrylamide gel it is frequently desirable to reduce the size of the polyacrylamide gel thereby obtaining gel particles. For example, the polyacrylamide gel may be cut into pieces having a diameter of 0.5 cm to 5 cm for dissolving the gel in water.

In one embodiment of the invention, the polyacrylamide gel is dissolved in water by means of a static mixer. For dissolving, a mixture of the polyacrylamide gel and water is fed into the static mixer.

The term “static mixer” is known in the art and refers to a mixer not comprising movable elements for mixing. Static mixers serve for continuous mixing of fluid materials. The energy needed for mixing comes from a loss in pressure as fluids flow through the static mixer. Mixing is accomplished through intense turbulence in the flow of the fluids to be mixed. Dissolving the polyacrylamide gel by means of a static mixer results in a homogenous aqueous polyacrylamide solution. It is to be noted that the term “static mixer” also covers an assembly of several static mixers. For example, two or more such as three static mixers may be arranged and connected to one another in a row. These static mixers serve comminuting purposes.

The polyacrylamide gel may be dissolved with a resting time within the static mixer of 0.05 s to 10 s and preferably 0.1 s to 2 s such as 1.0 s. The term “resting time within the static mixer” as used herein refers to the time it takes for a particle of the polyacrylamide gel to pass the mixer, i.e. from entering the mixer to being discharged therefrom. It is to be noted that the mixer discharges a kind of suspension which thickens and clears off within a few minutes such that the suspension physically dissolves after being passed through the mixer. Thus, a preferred viscosity for the aqueous polyacrylamide solution may be achieved due to rather minor degradation of the polymer.

Needless to say, the polyacrylamide gel may be dissolved by additional devices in combination with the static mixer such as mixer commercially available from Urschel Laboratories, Inc., 1200 Cutting Edge Drive, Chesterton, Ind. 46304 Unites States of America, for instance, the Comitrol® Processor Modell 1700, and/or by means of water jet cutting. For example, the polyacrylamide gel may be comminuted by an Urschel mixer and/or water jet cutting and subsequently completely dissolved by means of a static mixer.

The final concentration of the aqueous polyacrylamide solution may be selected by the skilled artisan according to the desired application. The polyacrylamide gel may be dissolved such that the aqueous polyacrylamide solution comprises 0.03% to 5.0% and preferably 0.05% to 2.0% by weight polyacrylamide. Thus, the aqueous polyacrylamide solution is well usable within mining or oil recovery.

The weight average molecular weight M_{w} of the polyacrylamide manufactured according to the present inventions is from 1.0*10⁶ g/mol to 50*10⁶ g/mol, preferably of 1.5*10⁶ g/mol to 30*10⁶ g/mol and more preferably 2.0*10⁶ g/mol to 25*10⁶ g/mol. The molecular weight can be determined for example by static light scattering, small angle neutron scattering, x-ray scattering or sedimentation velocity.

Typically, the polymers have intrinsic viscosity (IV), of at least 2 dl/g, for instance, from 2 to 40 dl/g, typically from 2 to 35 dl/g, suitably from 4 to 30 dl/g, frequently from 5 to 28 dl/g. Another suitable range may be from 3 to 12 dl/g, for instance, from 6 to 10 dl/g. Other suitable ranges include from 10 to 25 dl/g.

Intrinsic viscosity of polymers may be determined by preparing an aqueous solution of the polymer (0.5-1% w/w) based on the active content of the polymer. 2 g of this 0.5-1% polymer solution is diluted to 100 ml in a volumetric flask with 50 ml of 2M sodium chloride solution that is buffered to pH 7.0 (using 1.56 g sodium dihydrogen phosphate and 32.26 g disodium hydrogen phosphate per litre of deionised water) and the whole is diluted to the 100 ml mark with deionised water. The intrinsic viscosity of the polymers is measured using a Number 1 suspended level viscometer at 25° C. in 1M buffered salt solution. Intrinsic viscosity values stated are determined according to this method unless otherwise stated.

### Hydration of Acrylonitrile

The biocatalyst may encode the enzyme nitrile hydratase. With this regard, it is not relevant for the present invention whether the biocatalyst is naturally encoding nitrile hydratase, or whether it has been genetically modified to encode said enzyme, or whether a biocatalyst naturally encoding nitrile hydratase has been modified such as to be able to produce more and/or enhanced nitrile hydratase. As used herein, the term “biocatalyst encoding the enzyme nitrile hydratase” or the like generally means that such a biocatalyst is generally also able to produce and stably maintain nitrile hydratase. That is, as used herein and as readily understood by the skilled person, a biocatalyst, e.g. a microorganism, to be employed in accordance with the present invention which naturally or non-naturally encodes nitrile hydratase is generally also capable of producing and stably maintaining nitrile hydratase. However, in accordance with the present invention, it is also possible that such biocatalysts only produced nitrile hydratase during cultivation or fermentation of the biocatalyst—thus then containing nitrile hydratase—before being added to a reactor. Thus, in a preferred embodiment, the biocatalyst comprises nitrile hydratase. In such a case, it is possible that the biocatalysts do not produce nitrile hydratase during the methods described and provided herein any more, but they act only via the nitrile hydratase units which they have produced before and which they still contain. As readily understood by the person skilled in the art, it is also possible that some nitrile hydratase molecules may leave the biocatalyst, e.g. due to lysis of the microorganism, and act freely in the solution as biocatalyst. As such, it is also possible that the term “biocatalyst” as used herein encompasses the enzyme nitrile hydratase per se, as long as it is able to convert acrylonitrile to acrylamide as described and exemplified herein. In context with the present invention, it is also possible to directly employ nitrile hydratase as biocatalyst.

Accordingly, the biocatalyst may be alternatively or in addition a nitrile hydratase producing microorganism. In context with the present invention, microorganisms naturally encoding nitrile hydratase, which can be used as biocatalyst in any one of the methods described herein, comprise species belonging to a genus selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidiurn, Candida, Cryptococcus, Debaryomyces, Geotrichum, Hanseniaspora, Kluyveromyces, Pichia, Rhodotorula, Comomonas, and Pyrococcus. In preferred embodiments of the invention the biocatalyst is selected from bacteria of the genus Rhodococcus, Pseudomonas, Escherichia and Geobacillus.

Preferred biocatalysts to be employed in context with any one of the methods of the present invention comprise representatives of the genus Rhodococcus, e.g., Rhodococcus rhodochrous (e.g., NCIMB 41164, J1/FERM-BP 1478, M33 or M8), Rhodococcus pyridinovorans, Rhodococcus erythropolis, Rhodococcus equi, Rhodococcus ruber, or Rhodococcus opacus. Further species suitable as biocatalyst to be employed in context with any one of the methods of the present invention are, e.g., Aspergillus niger, Acidovorax avenae, Acidovorax facilis, Agrobacterium tumefaciens, Agrobacterium radiobacter, Bacillus subtilis, Bacillus pallidus, Bacillus smithii, Bacillus sp BR449, Bradyrhizobium oligotrophicum, Bradyrhizobium diazoefficiens, Bradyrhizobium japonicum, Burkholderia cenocepacia, Burkholderia gladioli, Escherichia coli, Geobacillus sp. RAPc8, Klebsiella oxytoca, Klebsiella pneumonia, Klebsiella variicola, Mesorhizobium ciceri, Mesorhizobium opportunistum, Mesorhizobium sp F28, Moraxella, Pantoea endophytica, Pantoea agglomerans, Pseudomonas chlororaphis, Pseudomonas putida, Rhizobium, Rhodopseudomonas palustris, Serratia liquefaciens, Serratia marcescens, Amycolatopsis, Arthrobacter, Brevibacterium sp CH1, Brevibacterium sp CH2, Brevibacterium sp R312, Brevibacterium imperiale, Brevibacterium casei, Corynebacterium nitrilophilus, Corynebacterium pseudodiphteriticum, Corynebacterium glutamicum, Corynebacterium hoffmanii, Microbacterium imperiale, Microbacterium smegmatis, Micrococcus luteus, Nocardia globerula, Nocardia rhodochrous, Nocardia sp 163, Pseudonocardia thermophila, Trichoderma, Myrothecium verrucaria, Aureobasidium pullulans, Candida famata, Candida guilliermondii, Candida tropicalis, Cryptococcus flavus, Cryptococcus sp UFMG-Y28, Debaryomyces hanseii, Geotrichum candidum, Geotrichum sp JR1, Hanseniaspora, Kluyveromyces thermotolerans, Pichia kluyveri, Rhodotorula glutinis, Comomonas testosteroni, Pyrococcus abyssi, Pyrococcus furiosus, or Pyrococcus horikoshii.

According to one embodiment of any one of the methods of the present invention, the biocatalyst to be employed belongs to the species Rhodococcus rhodochrous. Particular examples for strains belonging to Rhodococcus rhodochrous which may be employed in context with any one of the methods described herein comprise NCIMB 41164, J1 (FERM-BP 1478), M33 and M8.

Alternatively or in addition to Rhodococcus rhodochrous, the biocatalyst employed in any one of the methods described herein may be Rhodococcus pyridinovorans.

In context with the present invention, nitrile hydratase encoding microorganisms which are not naturally encoding nitrile hydratase may be genetically engineered microorganisms which naturally do not contain a gene encoding a nitrile hydratase but which have been manipulated such as to contain a polynucleotide encoding a nitrile hydratase (e.g., via transformation, transduction, transfection, conjugation, or other methods suitable to transfer or insert a polynucleotide into a cell as known in the art; cf. Sambrook and Russell 2001, Molecular Cloning: A Laboratory Manual, CSH Press, Cold Spring Harbor, N.Y., USA), thus enabling the microorganisms to produce and stably maintain the nitrile hydratase enzyme. For this purpose, it may further be required to insert additional polynucleotides which may be necessary to allow transcription and translation of the nitrile hydratase gene or mRNA, respectively. Such additional polynucleotides may comprise, inter alia, promoter sequences, polyT- or polyU-tails, or replication origins or other plasmid-control sequences. In this context, such genetically engineered microorganisms which naturally do not contain a gene encoding a nitrile hydratase but which have been manipulated such as to contain a polynucleotides encoding a nitrile hydratase may be prokaryotic or eukaryotic microorganisms. Examples for such prokaryotic microorganisms include, e.g., representatives of the species Escherichia coli. Examples for such eukaryotic microorganisms include, e.g., yeast (e.g., Saccharomyces cerevisiae).

In context of the present invention, the term “nitrile hydratase” (also referred to herein as NHase) generally means an enzyme which is capable of catalyzing the conversion (i.e. hydration) of acrylonitrile to acrylamide. Such an enzyme may be, e.g., the enzyme registered under IUBMB nomenclature as of Sep. 30, 2014: EC 4.2.1.84; CAS-No. 2391-37-5. However, the term “nitrile hydratase” as used herein also encompasses modified or enhanced enzymes which are, e.g., capable of converting acrylonitrile to acrylamide more quickly, or which can be produced at a higher yield/time-ratio, or which are more stable, as long as they are capable to catalyze conversion (i.e. hydration) of acrylonitrile to acrylamide. Methods for determining the ability of a given biocatalyst (e.g., microorganism or enzyme) for catalyzing the conversion of acrylonitrile to acrylamide are known in the art. As an example, in context with the present invention, activity of a given biocatalyst to act as a nitrile hydratase in the sense of the present invention may be determined as follows: First reacting 100 μl of a cell suspension, cell lysate, dissolved enzyme powder or any other preparation containing the supposed nitrile hydratase with 875 μl of an 50 mM potassium phosphate buffer and 25 μl of acrylonitrile at 25° C. on an eppendorf tube shaker at 1,000 rpm for 10 minutes. After 10 minutes of reaction time, samples may be drawn and immediately quenched by adding the same volume of 1.4% hydrochloric acid. After mixing of the sample, cells may be removed by centrifugation for 1 minute at 10,000 rpm and the amount of acrylamide formed is determined by analyzing the clear supernatant by HPLC. For affirmation of an enzyme to be a nitrile hydratase in context with the present invention, the concentration of acrylamide shall be between 0.25 and 1.25 mmol/l—if necessary, the sample has to be diluted accordingly and the conversion has to be repeated. The enzyme activity may then be deduced from the concentration of acrylamide by dividing the acrylamide concentration derived from HPLC analysis by the reaction time, which has been 10 minutes and by multiplying this value with the dilution factor between HPLC sample and original sample. Activities >5 U/mg dry cell weight, preferably >25 U/mg dry cell weight, more preferably >50 U/mg dry cell weight, most preferably >100 U/mg dry cell weight indicate the presence of a functionally expressed nitrile hydratase and are considered as nitrile hydratase in context with the present invention.

In context with the present invention, the nitrile hydratase may be a polypeptide encoded by a polynucleotide which comprises or consists of a nucleotide sequence which is at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99,5%, and most preferably 100% identical to the nucleotide sequence of SEQ ID NO: 1 (alpha-subunit of nitrile hydratase of R. rhodochrous: 5′- gtgagcgagcacgtcaataagtacacggagtacgaggcacgtaccaaggcgatcgaaaccttgctgtacgagcgagggctcatcacgcccgccgcggtcgaccgagtcgtttcgtactacgagaacgagatcggcccgatgggcggtgccaaggtcgtggccaagtcctgggtggaccctgagtaccgcaagtggctcgaagaggacgcgacggccgcgatggcgtcat tgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggt ttgggacagcagctccgaaatccgctacatcgtcatcccggaacggccggccggcaccgacggttggtccgaggaggagctgacgaagctggtgagccgggactcgatgatcggtgtcagtaatgcgctcacaccgcaggaagtgatcgtatga-3′) and/or to the nucleotide sequence of SEQ ID NO: 3 (beta-subunit of nitrile hydratase of R. rhodochrous: 5′- atggatggtatccacgacacaggcggcatgaccggatacggaccggtcccctatcagaaggacgagcccttcttccactacgagtgggagggtcggaccctgtcaattctgacttggatgcatctcaagggcatatcgtggtgggacaagtcgcggttcttccgggagtcgatggggaacgaaaactacgtcaacgagattcgcaactcgtactacacccactggctga gtgcggcagaacgtatcctcgtcgccgacaagatcatcaccgaagaagagcgaaagcaccgtgtgcaagagatccttgagggtcggtacacggacaggaagccgtcgcggaagttcgatccggcccagatcgagaaggcgatcgaacggcttcacgagccccactccctagcgcttccaggagcggagccgagtttctctctcggtgacaagatcaaagtgaagagtat gaacccgctgggacacacacggtgcccgaaatatgtgcggaacaagatcggggaaatcgtcgcctaccacggctgccagatctatcccgagagcagctccgccggcctcggcgacgatcctcgcccgctctacacggtcgcgttttccgcccaggaactgtggggcgacgacggaaacgggaaagacgtagtgtgcgtcgatctctgggaaccgtacctgatctctgcg tga-3′), provided that the polypeptide encoded by said polynucleotide is capable of catalyzing hydration of acrylonitrile to acrylamide (i.e. has nitrile hydratase activity) as described and exemplified herein. Also in the context with the present invention, the nitrile hydratase may be a polypeptide which comprises or consists of an amino acid sequence which is at least 70%, preferably at least 75%, more preferably at least 80%, more preferably at least 85%, more preferably at least 90%, more preferably at least 95%, more preferably at least 96%, more preferably at least 97%, more preferably at least 98%, more preferably at least 99%, more preferably at least 99,5%, and most preferably 100% identical to the amino acid sequence of SEQ ID NO: 2 (alpha-subunit of nitrile hydratase of R. rhodochrous: vsehvnkyte yeartkaiet llyerglitp aavdrvvsyy eneigpmgga kvvakswvdp eyrkwleeda taamaslgya geqahqisav fndsqthhvv vcticscypw pvlglppawy ksmeyrsrvv adprgvlkrd fgfdipdeve vrvwdsssei ryiviperpa gtdgwseeel tklvsrdsmi gvsnaltpqe viv, preferably: msehvnkyte yeartkaiet llyerglitp aavdrvvsyy eneigpmgga kvvakswvdp eyrkwleeda taamaslgya geqahqisav fndsqthhvv vcticscypw pvlglppawy ksmeyrsrvv adprgvlkrd fgfdipdeve vrvwdsssei ryiviperpa gtdgwseeel tklvsrdsmi gvsnaltpqe viv (SEQ ID NO:5)) and/or to the amino acid sequence of SEQ ID NO: 4 (beta-subunit of nitrile hydratase of R. rhodochrous: mdgihdtggm tgygpvpyqk depffhyewe grtlsiltwm hlkgiswwdk Srffresmgn enyvneirnsy ythwlsaae rilvadkiit eeerkhrvqe ilegrytdrk psrkfdpaqi ekaierlhep hslalpgaep sfslgdkikv ksmnplghtr cpkyvrnkig eivayhgcqi ypesssaglg ddprplytva fsaqelwgdd gngkdvvcvd lwepylisa), provided that said polypeptide is capable of catalyzing hydration of acrylonitrile to acrylamide as described and exemplified herein.

The level of identity between two or more sequences (e.g., nucleic acid sequences or amino acid sequences) can be easily determined by methods known in the art, e.g., by BLAST analysis. Generally, in context with the present invention, if two sequences (e.g., polynucleotide sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal of at least one nucleic acid residue or amino acid residue in a given sequence. The term “substitution” refers to the replacement of at least one nucleic acid residue/amino acid residue in a given sequence. Again, these definitions as used here apply, mutatis mutandis, for all sequences provided and described herein.

Generally, as used herein, the terms “polynucleotide” and “nucleic acid” or “nucleic acid molecule” are to be construed synonymously. Generally, nucleic acid molecules may comprise inter alia DNA molecules, RNA molecules, oligonucleotide thiophosphates, substituted ribooligonucleotides or PNA molecules. Furthermore, the term “nucleic acid molecule” may refer to DNA or RNA or hybrids thereof or any modification thereof that is known in the art (see, e.g., U.S. Pat. No. 5,525,711, U.S. Pat. No. 4,711,955, U.S. Pat. No. 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

As specified herein above, in a preferred embodiment, the term “nitrile hydratase” includes variants of the specifically indicated polynucleotides encoding at least one subunit of a nitrile hydratase. The term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N.Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2× SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, or plants preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))), which are part of the GCG software packet (Genetics Computer Group, 575 Science Drive, Madison, Wis., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention. The fragment shall still encode a polypeptide or fusion polypeptide which still has the activity as specified. Accordingly, the polypeptide encoded may comprise or consist of the domains of the polypeptide of the present invention conferring the said biological activity. A fragment as meant herein, preferably, comprises at least 50, at least 100, at least 250 or at least 450 consecutive nucleotides of any one of the specific nucleic acid sequences or encodes an amino acid sequence comprising at least 20, at least 30, at least 50, at least 80, at least 100 or at least 150 consecutive amino acids of any one of the specific amino acid sequences. The polynucleotides of the present invention either consist of, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein. The polynucleotide of the present invention shall be provided, preferably, either as an isolated polynucleotide (i.e. isolated from its natural context) or in genetically modified form. The polynucleotide, preferably, is DNA, including cDNA, or RNA. The term encompasses single as well as double stranded polynucleotides. Moreover, preferably, comprised are also chemically modified polynucleotides including naturally occurring modified polynucleotides such as glycosylated or methylated polynucleotides or artificial modified one such as biotinylated polynucleotides.

As specified herein above, in a preferred embodiment, the term “nitrile hydratase” includes variants of nitrile hydratase. As used herein, the term “polypeptide variant” relates to any chemical molecule comprising a polypeptide sequence of at least one subunit of a nitrile hydratase, preferably as specified elsewhere herein, said polypeptide variant having the indicated activity, but differing in primary structure from the nitrile hydratase indicated above. Thus, the polypeptide variant, preferably, is a mutein having the indicated activity. Preferably, the polypeptide variant comprises a peptide having an amino acid sequence corresponding to an amino acid sequence of 50 to 200, more preferably 60 to 175, even more preferably 70 to 150, or, most preferably, 80 to 130 consecutive amino acids comprised in a polypeptide as specified above. Moreover, also encompassed are further polypeptide variants of the aforementioned polypeptides. Such polypeptide variants have at least essentially the same biological activity as the specific polypeptides. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 50%, 60%, 70%, 80%, 85%, 90%, 92%, 95%, 97%, 98%, or 99% identical with the amino acid sequence of the specific polypeptide. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the sequence it is compared to for optimal alignment. The percentage is calculated by determining, preferably over the whole length of the polypeptide, the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Polypeptide variants referred to herein may be allelic variants or any other species specific homologs, preferably a homolog from one of the microorganisms as specified above, paralogs, or orthologs. Moreover, the polypeptide variants referred to herein include fragments of the specific polypeptides or the aforementioned types of polypeptide variants as long as these fragments and/or variants have the biological activity as referred to above. Such fragments may be or be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics.

When adding the biocatalyst to the reactor in any one of the methods of the present invention, the biocatalyst may be taken directly from the fermentation broth. It is further envisaged that the biocatalyst may be employed in the form of a fermentation broth in the methods disclosed herein. Thus, the biocatalyst does not need to be isolated from the fermentation broth, and a fermentation broth comprising the biocatalyst may be used for the bioconversion. For example, a fermentation broth comprising the biocatalyst may be added to the reactor of the methods of the present invention. Alternatively, in accordance with any one of the methods described herein, the biocatalyst may have been dried before being added to the reactor. In this context the term “before” does not necessarily mean that the biocatalyst has been dried and is then directly added to the reactor. It is rather sufficient that the biocatalyst has undergone a drying step at any time before it is added to the reactor, independently of whether further steps between the drying and the addition are performed or not. As non-limiting examples, such further steps between the drying step and the addition to the reactor may be storage or reconstitution. However, it is also possible to add the biocatalyst to the reactor directly after drying. The inventors have surprisingly found that by using a biocatalyst, which has undergone a drying step, the concentration of acrylic acid in an aqueous acrylamide solution obtained by any one of the methods described herein is further reduced in comparison to the case that a biocatalyst is used which has not undergone drying before being employed in the bioconversion.

Regarding the drying method, in any one of the methods described an provided herein, a biocatalyst may be used which has been dried using freeze-drying, spray drying, heat drying, vacuum drying, fluidized bed drying and/or spray granulation. With this respect, spray drying and freeze drying are preferred, since in general by using a biocatalyst, which has been subjected to spray- or freeze drying, a higher reduction of the acrylic acid concentration in the obtained aqueous acrylamide solutions is achieved compared to employing a biocatalyst which has been dried using other methods.

A conversion of acrylonitrile to acrylamide may be carried out with a so as to obtain an acrylamide solution with a concentration of 25% to 45% by weight acrylamide monomers. The concentration of acrylamide in the obtained solution is preferably in the range from 20% to 80%, more preferably in the range from 30% to 70%, most preferably in the range from 40% to 60% by weight of acrylamide monomers.

The biocatalyst may be removed before the polymerization of the acrylamide solution to polyacrylamide gel is carried out. For example, the biocatalyst may be removed by means of filtration. Thus, any deterioration of the polyacrylamide due to encapsulation of the biocatalyst is avoided. Separation of the biocatalyst may take place by for example filtration or centrifugation. Preferred may also be the use of active carbon for separation purpose. Such a removal or separation process step is carried out in-line. For example, a filter may be provide in a line or pipe connecting a first reactor for carrying out the conversion of acrylonitrile to acrylamide and a second reactor for carrying out the polymerization of the acrylamide solution.

A conversion of acrylonitrile to acrylamide may be carried out at a starting temperature of 15° C. to 30° and preferably of 20° C. to 25° C. The polymerization of the acrylamide solution to polyacrylamide gel may be carried out at a temperature of 0° C. to 20° and preferably of 2° C. to 5° C. It is to be noted that the conversion of acrylonitrile to acrylamide is an adiabatic process wherein the temperature during is process raises up to 100° C. and particularly 80° C. to 95° C.

### Gel Polymerization

Polymerization of the aqueous monomer solution comprising acryl amide and optionally further monoethylenically unsaturated, water-soluble monomers is performed by radical polymerization by the gel polymerization technique, preferably adiabatic gel polymerization. In gel polymerization a relatively concentrated solution of monomers in an aqueous solvent is polymerized thereby obtaining a polymer gel. The polymerization mixture is not stirred during polymerization because the stirrer would stick in course of polymerization.

The aqueous monomer solution to be polymerized should comprise at least 10% by weight of acryl amide and optionally further water-soluble monomers. The aqueous monomer solution may comprise 16% to 50% by weight of monomers, preferably 18% to 48%, more preferably 20% to 45% even more preferably 25% to 40% and still more preferably 32% to 38%.

In one embodiment, acrylic acid and/or 2-acrylamido-2-methylpropane sulfonic acid and/or their respective salts are present, thereby obtaining a polyacrylamide solution comprising 25% to 40% by weight, preferably of 26% to 39% by weight and more preferably 27% to 38% by weight of acrylic acid and/or 2-acrylamido-2-methylpropane sulfonic acid.

The polymerization of the acrylamide may in particular be initiated by addition of an initiator for radical polymerization.

The radical polymerization initiator may be added with a concentration of 0.01% to 5.0% by weight and preferably of 0.02% to 2.0% by weight relating to the total weight of its solution.

The radical polymerization initiator may be selected from the group of peroxides, persulfates, azo compounds, redox couples and mixtures thereof.

Examples of peroxides are hydrogen peroxide, potassium peroxide, tert-butyl peroxide, tert-butyl hydroperoxide, cumene hydroperoxide and benzoyl peroxide. Examples of persulfates are ammonium, sodium or potassium persulfate. Examples of azo compounds are 2,2-azo-bisisobutyronitrile, 4,4′-azobis(4-cyanovaleric acid) and 2,2′-azobis(N,N-dimethyleneisobutyramidine) dihydrochloride, 1,1′-azobis(cyclohexanecarbonitrile) and 2,2′-azobis(2-amidino-propane) dihydrochloride. Redox couples consist of an oxidizing agent and a reducing agent. The oxidizing agent can be one of the above listed peroxides, persulfates, or an alkali metal chlorate or bromate. Examples of reducing agents are ascorbic acid, glucose or ammonium or alkali metal hydrogen sulfite, sulfite, thiosulfate or sulfide, or ferrous ammonium sulfate. Redox initiators are capable of initiating radical polymerization already at low temperatures, e.g. already at temperatures of 5° C. or less.

Preferably, the radical polymerization initiator is a mixture of a redox couple with one or more radical polymerization initiators different from redox couples, preferably azo compounds.

More preferably, the initiator is a mixture of a redox couple, wherein the oxidizing agent is selected from the group consisting of peroxides and alkali metal bromates, and the reducing agent is selected from the group consisting of ammonium or alkali metal hydrogen sulfite, sulfite, thio-sulfate or sulfide, or ferrous ammonium sulfate, with one or more azo compound initiators.

Even more preferably, the initiator is a mixture of a redox couple, wherein the oxidizing agent is selected from the group consisting of hydrogen peroxides and alkali metal bromates, and the reducing agent is an alkali metal hydrogen sulfite or sulfite, with one or more azo compound initiators.

Most preferably, the initiator is a mixture of a redox couple, wherein the oxidizing agent is selected from the group consisting of tert-butylhydroperoxide and potassium bromate, and the reducing agent is sodium sulfite, with one or more azo compound initiators selected from the group consisting of 2,2-azobisisobutyronitrile, 4,4′-azobis(4-cyanovaleric acid) and 2,2′-azo-bis(N,N-dimethyleneisobutyramidine).

Redox initiators may thus be based on Fe²⁺/Fe³⁺—H₂O₂, Fe²⁺/Fe³⁺-alkylhydroperoxide, alkylhydroperoxides-sulfite, e.g. t-butylhydroperoxide-sodiumsulfite, peroxides-thiosulfate or alkylhydroperoxide-sulfonates, e.g. alkylhydroperoxide/hydroxymethansulfinates, e.g. t-butylhydroperoxide-sodiumhydroxymethansulfinate.

Adding of the radical polymerization initiator(s) is carried out immediately before polymerization. A solution such as an aqueous solution of the radical polymerization initiator is preferably used. Such a solution may be supplied during or after filling of a polymerization reactor. Preferably, the solution is supplied to the monomers during filling of the polymerization reactor. In order to accelerate mixing of the radical polymerization initiator(s) and the aqueous monomer solution, the monomer supply may be equipped with a static mixer.

The polymerization preferably is conducted under adiabatic conditions. “Adiabatic” is understood by the person skilled in the art to mean that there is no exchange of heat with the environment. This ideal is naturally difficult to achieve in practical chemical engineering. In the context of this invention, “adiabatic” shall consequently be understood to mean “essentially adiabatic”, meaning that the reactor is not supplied with any heat from the outside during the polymerization, i.e. is not heated, and the reactor is not cooled during the polymerization. However, it will be clear to the person skilled in the art that—according to the internal temperature of the reactor and the ambient temperature—certain amounts of heat can be released or absorbed via the reactor wall because of temperature gradients, but this effect naturally plays an ever lesser role with increasing reactor size.

The adiabatic gel polymerization is started at ambient temperatures or below. The initiation temperature of the polymerization is less than 5° C., preferably −4° C. to +4° C., more preferably −4° C. to 0° C. For achieving such temperatures, the monomer solution needs to be cooled. Such cooling preferably is performed before aqueous monomer solution comprising acryl amide and optionally further monoethylenically, water-soluble monomers is filled into the polymerization reactor. For initiating the polymerization at least one redox initiator is used. Preferably, a solution of the redox initiator is fed into the monomer supply line comprising the cooled monomer solution directly before the supply line enters into the reactor. Mixing may be supported by means of a static mixer.

The polymerization starts even at such low temperatures because of the redox initiator(s) added. The heat of polymerization released heats up the mixture. Under the influence of the heat of polymerization evolved, the polymerization mixture heats up to a temperature of 60° C. to 100° C.

Preferably, a mixture of at least one redox initiator and an azo initiator is used. Suitable mixtures and preferred mixtures have already been mentioned above. Polymerization starts upon addition of the redox initiator. On attainment of a sufficient temperature, the azo initiator(s) also begin to break down and likewise initiate the polymerization.

After the polymerization, the polymer gel formed can be withdrawn from the reactor. This can be effected by means of mechanical auxiliaries, for example with the aid of a ram in the case of a tubular reactor. In addition, the reactor may have outlet valves arranged at the base, and the polyacrylamide gel can be expressed from the reactor with the aid of gases such as compressed air or nitrogen.

The method may be monitored on line. Thus, the complete process of the preparation of the aqueous polyacrylamide solution may be supervised. Thereby, a target quality of the aqueous polyacrylamide solution may be ensured.

The method may be carried out on site. The term “on site” as used herein refers to an actual site where the polyacrylamide solution is to be used or closely adjacent thereto. Thus, instead of expensive preparation of dry polyacrylamide and transportation to the actual site of use, where the polyacrylamide has to be dissolved and diluted, significant costs may be saved with the method according to the present invention.

In one embodiment of the invention, the method is carried out on an oilfield and the polyacrylamide solution manufactured is used for oilfield applications, in particular for enhanced oil recovery.

In another embodiment of the invention, the method is carried out on in a mining area and the polyacrylamide solution manufactured is used for mining applications.

The method may be carried out in at least one mobile reactor. Thus, the polyacrylamide solution may be produced exactly with quantities as demanded. Further, the aqueous polyacrylamide solution may be transferred after being dissolved to the position on site, where it is to be used. Thus, pumps and long pipes may be avoided but the complete method bay be carried out where demanded in a flexible manner.

The method may be carried out in a time of 12 h to 72 h and preferably of 15 h to 60 h. Thus, the prepared aqueous polyacrylamide solution is ready to be used within a rather short time.

The aqueous polyacrylamide solution may be prepared so as to be suitable in oil recovery and for mining. Thus, the method according to the present invention may be carried out in a flexible manner concerning the site for the preparation and the quantity of the aqueous polyacrylamide solution.

Summarizing the above, the method according to the present invention provides advantages as it is configured for an energy saving, compact and transportable installation for on-site production of polyacrylamide or copolymers of acrylamide via gel free radical polymerization starting with acrylonitrile as raw material. All the process steps are run at ambient temperatures without any heating and without the need for energy intensive processing steps like granulation, grinding, drying, concentration, evaporation and without addition of any chemicals for processing like lubricants, anti-sticking material, or the like and without dust generation. Especially the current practice in the industry to first remove the water present in the polymer gel in order to save transportation cost and later on to add water back to dissolve the polymer is completely overcome by a scalable, on purpose onsite polymer solution production method.

### SHORT DESCRIPTION OF THE FIGURES

Further features and embodiments of the invention will be disclosed in more detail in the subsequent description of embodiments, particularly in conjunction with the dependent claims. Therein, the respective features may be realized in an isolated fashion as well as in any arbitrary feasible combination, as the skilled person will realize. The scope of the invention is not restricted by the embodiments. The embodiments are schematically depicted in the figures. Therein, identical reference numbers in these figures refer to identical or functionally comparable elements.

In the figures:

FIG. 1 shows a block diagram of an installation for the preparation of a polyacrylamide solution.

FIG. 2 schematically shows a polymerization reactor having a tubular part and a conical taper at its lower end.

FIG. 3 schematically shows a polymerization reactor having a conical part and a second conical taper at its lower end.

### DETAILED DESCRIPTION OF THE EMBODIMENTS

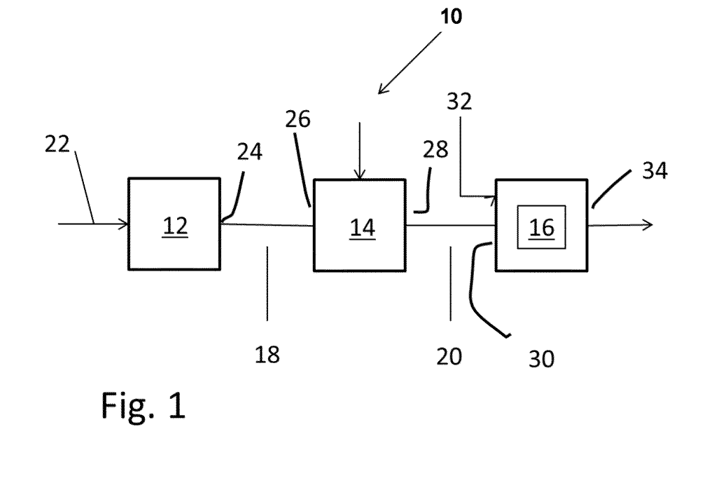

FIG. 1 shows a block diagram of an installation 10 for preparing of a polyacrylamide solution. The installation 10 basically comprises at least one reactor for preparing acrylamide from acrylonitrile, one reactor for polymerizing the aqueous monomer solution comprising acrylamide and optionally further monoethylenically unsaturated, water-soluble monomers and a device for dissolving the polyacrylamide gel to an aqueous polyacrylamide solution as will be explained in further detail hereinafter.

According to the exemplary embodiment shown in FIG. 1, the installation 10 comprises a first reactor 12, a second reactor 14 and a static mixer 16. The first reactor 12 is connected to the second reactor 14 by means of a pipe 18. The second reactor 14 is connected to the static mixer 16 by means of a pipe 20. The installation 10 is configured to be used with a method for preparing of an aqueous polyacrylamide solution as will be explained in further detail hereinafter.

The first reactor 12 comprises at least one feed 22. By means of the feed 22, water and acrylonitrile are supplied to the first reactor 12. Further, a biocatalyst is supplied to the first reactor 12. The acrylonitrile is hydrated in the water in presence of the biocatalyst. The biocatalyst is capable of converting acrylonitrile to acrylamide so as to obtain an acrylamide solution. The biocatalyst encodes the enzyme nitrile hydratase. For this purpose, the biocatalyst is a nitrile hydratase producing microorganism. For example, the nitrile hydratase producing microorganism is a species belonging to a genus selected from the group consisting of Rhodococcus, Aspergillus, Acidovorax, Agrobacterium, Bacillus, Bradyrhizobium, Burkholderia, Escherichia, Geobacillus, Klebsiella, Mesorhizobium, Moraxella, Pantoea, Pseudomonas, Rhizobium, Rhodopseudomonas, Serratia, Amycolatopsis, Arthrobacter, Brevibacterium, Corynebacterium, Microbacterium, Micrococcus, Nocardia, Pseudonocardia, Trichoderma, Myrothecium, Aureobasidium, Candida, Cryptococcus, Debaryomyces, Geotrichum, Hanseniaspora, Kluyveromyces, Pichia, Rhodotorula, Comomonas, and Pyrococcus. In preferred embodiments of the invention the biocatalyst is selected from bacteria of the genus Rhodococcus, Pseudomonas, Escherichia and Geobacillus. Preferred biocatalysts to be employed in context with the method of the present invention comprise representatives of the genus Rhodococcus. Species suitable as biocatalyst to be employed in context with any one of the method of the present invention may comprise, e.g., Rhodococcus rhodochrous. In order to increase the contact of the acrylonitrile and the biocatalyst, a stirrer (not shown in detail) may be present within the first reactor 12. As a biocatalyst is used for converting acrylonitrile to acrylamide, the conversion is carried out at a temperature of 15° C. to 30° and preferably of 20° C. to 25° C. Thus, a heating for initiating the conversion is not necessary. Rather, the conversion may be carried out at ambient temperature. For example, the conversion is carried out at a temperature of 22° C. The amount of biocatalyst used for the conversion process depends on the concentration of the acrylamide solution to be produced within a target time. Thus, the higher the target concentration of the acrylamide solution is the more biocatalyst is used in order to produce this acrylamide amount in the same time as with a lower concentration.

The thus formed acrylamide solution is directly supplied to the second reactor 14. For example, the acrylamide solution may be discharged from the first reactor 12 through an outlet 24 thereof and is supplied to the second reactor 14 through the pipe 18 and a feed 26 of the second reactor 14. It is to be noted that a buffer tank (not shown in detail) may be disposed between the first reactor 12 and the second reactor 14 fur buffering the acrylamide solution before being supplied to the second reactor 14 if technically required. For example, a buffer tank, which is configured to contain an amount or volume corresponding to at least the target amount or target volume of the acrylamide solution supplied to the second reactor 14, may be disposed between the first reactor 12 and the second reactor 14. Thus, the buffer tank may buffer one filling amount or volume of the second reactor 14. The biocatalyst may be removed from the acrylamide solution. For example, a filter (not shown in detail) may be present within the pipe 18 configured to hold back the biocatalyst. Within the second reactor 14, the acrylamide solution is directly polymerized so as to obtain a polyacrylamide gel. The polymerization of the acrylamide is initiated by addition of a radical polymerization initiator. The radical polymerization initiator may be added with a concentration of 0.01% to 5.0% by weight and preferably of 0.02% to 2.0% by weight relating to the total weight of its solution such as 0.1%. The radical polymerization initiator may be selected from the group of peroxides, persulfates, azo compounds, redox couples and mixtures thereof. Suitable examples have already been provided above.

The polymerization of the acrylamide solution to polyacrylamide gel preferably may be carried out under adiabatic conditions. Details have already been mentioned above.

The polymerization may be performed in any kind of reactor suitable for gel polymerization. Such reactors are basically known to the skilled artisan. Particularly advantageously, it is possible to use conical reactors for this purpose, as described, for example, by U.S. Pat. No. 5,633,329 or U.S. Pat. No. 7,619,046 B2.

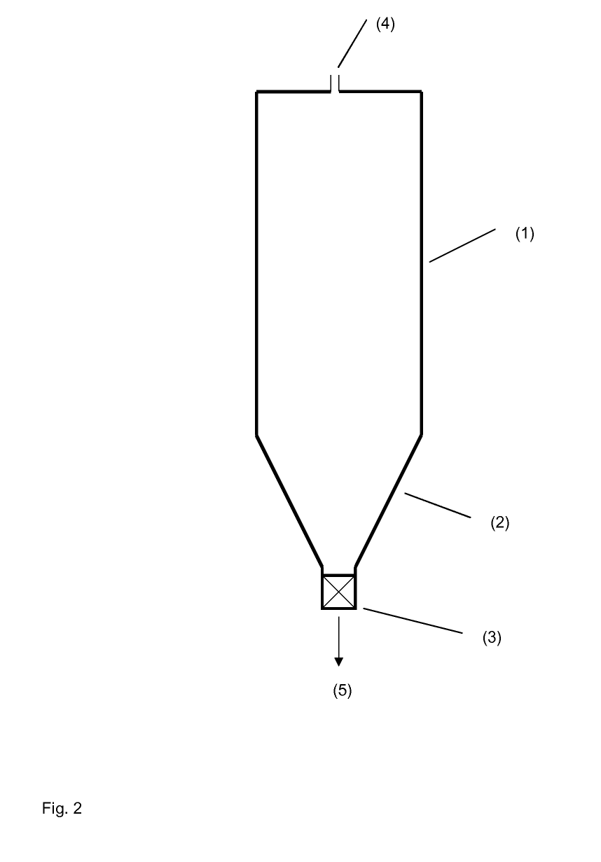

FIG. 2 schematically shows vertical tubular reactor (1) which narrows conically (2) at the lower end. The capacity of the reactors is chosen by the person skilled in the art according to the desired production capacity and may be 1 to 100 m³, for example 5 to 50 m³, without any intention that the invention be restricted thereto. The inner surface of the reactor has preferably been provided with a coating to reduce the adhesion of the reaction mixture to the reactor wall, for example with a Teflon coating. At the lower end, the reactor has a shut-off device (3). The reactor further comprises at least one feed (4). Through this feed (4), the aqueous monomer solution and/or gases and/or further components can be passed into the reactor. Gases may especially be inert gases such as nitrogen, argon or CO₂. Inert gases can be used to purge the reactor for inertization. Of course, it is also possible for different feeds to be present for different components, for example separate feeds for the aqueous reaction solution and gases. The at least one feed (4) may preferably be mounted at the top of the reactor or at the side in the upper region of the reactor, but other arrangements are of course also possible.

The shut-off device (3) is closed during polymerization. To withdraw the polymer gel from the reactor, the shut-off device (3) is opened. In general, the polymer gel obtained is such viscous that it does not flow out of the reactor without additional measures. For removing the polyacrylamide gel (5) a gas such as nitrogen, pressurized air or carbon dioxide or a liquid, in particular water is injected at the top of the tubular reactor via the feed (4) or another feed, thereby pressing the polyacrylamide gel out of the reactor. The shut-off device may be connected with a screw conveyor or some other conveyor which transfers the polyacrylamide gel to the device for dissolving. Such a screw conveyor may also support removing the gel from the polymerization reactor. Furthermore, it already causes some comminution of the polyacrylamide gel.

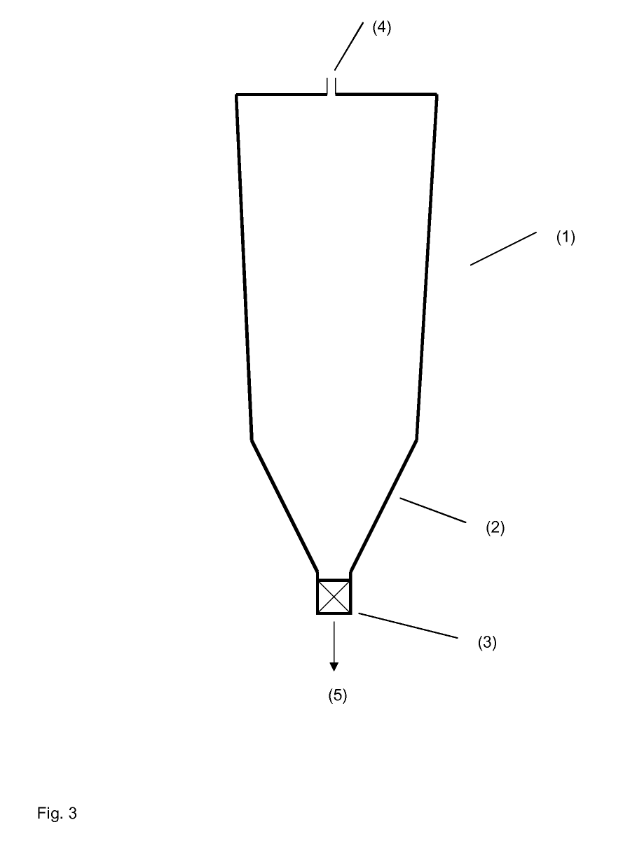

FIG. 3 shows another embodiment of a conical reactor. In this embodiment, the upper part is not tubular but also slightly conical, i.e. the reactor comprises two different conical sections. Besides that, the function of the reactor is the same.

In the exemplary embodiment according to FIG. 1, the thus formed polyacrylamide gel is directly supplied to the static mixer 16. For example, the polyacrylamide gel may be discharged from the second reactor 14 through an outlet 28 (for example the bottom outlet as indicated in FIG. 1 or 2) thereof and is supplied to the static mixer 16 through the pipe 20 and a feed 30 of the static mixer 16. The polyacrylamide gel is directly dissolved by addition of water so as to obtain an aqueous polyacrylamide solution by means of the static mixer. The water may be added through a separate feed 32 of the static mixer 16. The polyacrylamide gel is dissolved with a resting time within the mixer of 0.05 s to 10 s and preferably 0.1 s to 2 s such as 1.0 s. The aqueous polyacrylamide solution may be discharged from the static mixer 16 through an outlet 34. The polyacrylamide gel is dissolved such that the aqueous polyacrylamide solution comprises 0.03% to 5.0% and preferably 0.05% to 2.0% by weight polyacrylamide such as 1.0%. Thus, the aqueous polyacrylamide solution is suitable in mining and/or oil recovery.

In another embodiment, the static mixer may comminute the gel and partly dissolve it, thereby obtaining a mixture of water, polyacrylamide already dissolved therein and particles of polyacrylamide gel not yet dissolved. The process of dissolving may be finalized in a vessel, for example a stirred vessel or by passing the mixture through a second static mixer.

The method is carried out in a time of 12 h to 72 h and preferably of 15 h to 60 h such as 20 h. For example, the step of converting acrylonitrile to acrylamide may be carried out such that it takes 4 h to 8 h and preferably 6 h to 7 h so as to provide an acrylamide solution comprising 50% acrylamide. In order to produce 1 t acrylamide solution with a concentration of 50% by weight acrylamide, 0.1 kg to 1.0 kg, preferably 0.16 kg to 0.75 kg and more preferably 0.2 kg to 0.6 kg biocatalyst is used. The biocatalyst may be used as a dried powder such as dried by means of spray drying. If the target concentration within the same time is lower, the amount of biocatalyst may be linearly reduced. For example, if the target concentration of the acrylamide solution is 30% by weight acrylamide, 0.06 kg to 0.6 kg, preferably 0.10 kg to 0.45 kg and more preferably 0.13 kg to 0.36 kg biocatalyst is used per ton acrylamide solution. If the target concentration of the acrylamide solution is 35% by weight acrylamide, 0.07 kg to 0.7 kg, preferably 0.11 kg to 0.53 kg and more preferably 0.15 kg to 0.42 kg biocatalyst is used per ton acrylamide solution. If the target concentration of the acrylamide solution is 40% by weight acrylamide, 0.08 kg to 0.8 kg, preferably 0.13 kg to 0.60 kg and more preferably 0.17 kg to 0.48 kg biocatalyst is used per ton acrylamide solution.

Needless to say, the step of the conversion of acrylonitrile to acrylamide is carried out with a speed that is adapted to the speed of the polymerizing step. Thus, it is ensured that the polymerization step is entered with exactly the amount of acrylamide that is formable by the conversion of acrylonitrile to acrylamide. This avoids the provision of storage tanks for storing acrylamide and the method may be continuously carried out. For example, the step of polymerizing acrylamide to polyacrylamide may be carried out such that it takes 4 h to 8 h and preferably 6 h to 7 h so as to provide a polyacrylamide gel with a concentration of 25% to 40% by weight, preferably of 26% to 39% by weight and more preferably 27% to 38% by weight acrylamide within the polyacrylamide gel in water such as 30%.

The method may be monitored on line. Further, may be carried out on site. Thus, the installation 10 may be disposed at a site where the polyacrylamide solution is actually used, for example at an oilfield or at a mining area. The at least one reactor may be mobile. For example, the above described first and second reactors 12, 14 may be mobile and disposed on a vehicle. Needless to say, the static mixer 16 may be mobile as well such that the complete installation 10 may be mobile.

Basically, by means of the disclosed method, water-soluble homo- or copolymers of (meth)acrylamide by free-radical polymerization are provided as an aqueous solution. In this process, acrylamide or methacrylamide is obtained from acrylonitrile or methacrylonitrile and includes monomers in aqueous solution in a comparatively high concentration, namely 25 to 45% by weight. Because of the high concentration, the mixture does not remain liquid in the course of the polymerization; instead, a solid, water-containing polymer gel is obtained.

### Homo- and Copolymers of Acryl Amide to be Manufactured

Accordingly, by means of the process according to the invention, it is possible to prepare water-soluble homo- or copolymers of (meth)acrylamide. They comprise monoethylenically unsaturated, hydrophilic monomers (A1), where at least one of the monomers is (meth)acrylamide. Optionally, monoethylenically unsaturated, amphiphilic monomers (A2) other than the hydrophilic monomers (A1) and further ethylenically unsaturated monomers (A3) may be present.

The monoethylenic monomers (A1) are hydrophilic. The term “hydrophilic” in the context of this invention means that the monomers (A) are to be soluble in the aqueous acrylamide solution to be used for polymerization, i.e. a solution comprising 25 to 45% by weight of monomers (A1), in the desired use concentration. It is thus not absolutely necessary that monomers (A) to be used are miscible with water without any gap; instead, it is sufficient if they meet the minimum requirement mentioned. In general, the solubility of the hydrophilic monomers (A) in water at room temperature should be at least 50 g/l, preferably at least 100 g/l and more preferably at least 150 g/l.

The hydrophilic, monoethylenically unsaturated monomers (A1) may be uncharged monomers (A1a). The monomers (A1a) comprise hydrophilic groups which impart at least a certain water solubility to the monomers. (Meth)acrylamide is a monomer (A1a). Examples of further monomers (A1a) include derivatives of (meth)acrylamide such as N-methyl(meth)acrylamide, N,N′-dimethyl(meth)acrylamide or N-methylol(meth)acrylamide.

Further examples include monomers comprising hydroxyl and/or ether groups, for example hydroxyethyl (meth)acrylate, hydroxypropyl (meth)acrylate, allyl alcohol, hydroxyvinyl ethyl ether, hydroxyvinyl propyl ether, hydroxyvinyl butyl ether, polyethylene glycol (meth)acrylate, N-vinylformamide, N-vinylacetamide, N-vinylpyrrolidone or N-vinylcaprolactam, and vinyl esters, for example vinyl formate or vinyl acetate. N-Vinyl derivatives can be hydrolyzed after polymerization to give vinylamine units, and vinyl esters to give vinyl alcohol units.

Hydrophilic, monoethylenically unsaturated monomers (A1) may be hydrophilic, anionic monomers (A1b) comprising at least one acidic group, or salts thereof.

The acidic groups are preferably acidic groups selected from the group of —COOH, —SO₃H and —PO₃H₂ or salts thereof. Preference is given to monomers comprising COOH groups and/or —SO₃H groups, particular preference to monomers comprising —SO₃H groups. The salts of the acidic monomers may of course also be involved. Suitable counterions include especially alkali metal ions such as Li⁺, Na⁺ or K⁺, and also ammonium ions such as NH₄⁺ or ammonium ions having organic radicals. Examples of ammonium ions having organic radicals include [NH(CH₃)₃]⁺, [NH₂(CH₃)_{2]}⁺, [NH₃(CH₃)]⁺, [NH(C₂H₅)_{3]}⁺, [NH₂(C₂H₅)₂]⁺, [NH₃(C₂H₅)]⁺, [NH₃(CH₂CH₂OH)]⁺, [H₃N—CH₂CH₂—NH₃]²⁺ or [H(H₃C)₂N—CH₂CH₂CH₂NH₃]²⁺.

Examples of monomers (A1b) comprising COOH groups include acrylic acid, methacrylic acid, crotonic acid, itaconic acid, maleic acid or fumaric acid. Preference is given to acrylic acid.

Examples of monomers (A1b) comprising sulfo groups include vinylsulfonic acid, allylsulfonic acid, 2-acrylamido-2-methylpropanesulfonic acid, 2-methacrylamido-2-methylpropanesulfonic acid, 2-acrylamidobutanesulfonic acid, 3-acrylamido-3-methylbutanesulfonic acid or 2-acrylamido-2,4,4-trimethylpentanesulfonic acid. Preference is given to vinylsulfonic acid, allylsulfonic acid or 2-acrylamido-2-methylpropanesulfonic acid and particular preference to 2-acrylamido-2-methylpropanesulfonic acid (APMS) or salts thereof.

Examples of monomers (A1b) comprising phosphonic acid groups include vinylphosphonic acid, allylphosphonic acid, N-(meth)acrylamidoalkylphosphonic acids or (meth)acryloyloxyalkyl-phosphonic acids, preferably vinylphosphonic acid.

Preferably, monomer (A1b) may be selected from the group consisting of acrylic acid, methacrylic acid, crotonic acid, itaconic acid, maleic acid, fumaric acid, vinylsulfonic acid, allylsulfonic acid, 2-acrylamido-2-methylpropanesulfonic acid (AMPS), 2-methacrylamido-2-methylpropane-sulfonic acid, 2-acrylamidobutanesulfonic acid, 3-acrylamido-3-methylbutane-sulfonic acid, 2-acrylamido-2,4,4-trimethylpentanesulfonic acid, vinylphosphonic acid, allylphosphonic acid, N-(meth)acrylamidoalkylphosphonic acids and (meth)acryloyloxyalkyl-phosphonic acids, more preferably from acrylic acid and/or APMS or salts thereof.

Further, monoethylenically unsaturated, hydrophilic monomers may be hydrophilic, cationic monomers (A1c). Suitable cationic monomers (A1c) include especially monomers having ammonium groups, especially ammonium derivatives of N-(ω-aminoalkyl)(meth)acrylamides or ω-aminoalkyl (meth)acrylates.

More particularly, monomers (A1c) having ammonium groups may be compounds of the general formulae H₂C═C(R¹)—CO—NR²—R³—N(R⁴)₃+ X⁻ (Ia) and/or H₂C═C(R¹)—COO—R³—N(R⁴)₃+ X⁻ (Ib). In these formulae, R¹ is H or methyl, R² is H or a C₁— to C₄-alkyl group, preferably H or methyl, and R⁴ is a preferably linear C₁— to C₄-alkylene group, for example a 1,2-ethylene group —CH₂—CH₂— or a 1,3-propylene group —CH₂—CH₂—CH₂—. The R⁴ radicals are each independently C₁- to C₄-alkyl radicals, preferably methyl or a group of the general formula —R⁵—SO₃H where R⁵ is a preferably linear C₁— to C₄-alkylene group or a phenyl group, with the proviso that generally not more than one of the R⁴ substituents is a substituent having sulfo groups. More preferably, the three R⁴ substituents are methyl groups, meaning that the monomer has an —N(CH₃)₃₊ group. X⁻ in the above formula is a monovalent anion, for example Cl⁻. X⁻ may of course also be a corresponding fraction of a polyvalent anion, although this is not preferred. Examples of preferred monomers (A1c) of the general formula (Ia) or (Ib) include salts of 3-trimethylammoniopropyl(meth)acrylamides or 2-trimethylammonioethyl (meth)acrylates, for example the corresponding chlorides such as 3-trimethylammoniopropylacrylamide chloride (DIMAPAQUAT) and 2-trimethylammonioethyl methacrylate chloride (MADAME-QUAT).

The amphiphilic monomers (A2) are monoethylenically unsaturated monomers having at least one hydrophilic group and at least one, preferably terminal, hydrophobic group. Monomers of this kind serve to impart hydrophobically associating properties to copolymers comprising (meth)acrylamide.

“Hydrophobically associating copolymers” are understood by the person skilled in the art to mean water-soluble copolymers which, as well as hydrophilic units (in a sufficient amount to assure water solubility), have hydrophobic groups in lateral or terminal positions. In aqueous solution, the hydrophobic groups can associate with one another. Because of this associative interaction, there is an increase in the viscosity of the aqueous polymer solution compared to a polymer of the same kind that merely does not have any associative groups.

Suitable monomers (A2) especially have the general formula H₂C═C(R⁵)—R⁶—R⁷ (IIa) where R⁵ is H or methyl, R⁶ is a linking hydrophilic group and R⁷ is a terminal hydrophobic group. In a further embodiment, the monomer (A2) may have general formula H₂C═C(R⁵)—R⁶—R⁷—R⁸ (IIb) where R⁵,

R⁶ and R⁷ are each as defined above, and R⁸ is a hydrophilic group.

The linking hydrophilic R⁶ group may be a group comprising alkylene oxide units, for example a group comprising 5 to 50 alkylene oxide units, which is joined to the H₂C═C(R⁵) group in a suitable manner, for example by means of a single bond or of a suitable linking group, where at least 70 mol %, preferably at least 90 mol %, of the alkylene oxide units are ethylene oxide units. In addition, the group may be a group comprising quaternary ammonium groups.

In one embodiment of the invention, the hydrophobic R⁷ group comprises aliphatic and/or aromatic, straight-chain or branched C₈₋₄₀-hydrocarbyl radicals R^{7a}, preferably C₁₂₋₃₂-hydrocarbyl radicals. In a further embodiment, the hydrophobic R⁷ group may be an R^{7b} group comprising alkylene oxide units having at least 3 carbon atoms, preferably at least 4 carbon atoms.

In one embodiment of the invention, the monomers (A2) are monomers of the general formula

H₂C═C(R⁵)—O—(—CH₂—CH(R⁸)—O—)ₖ— (IIc) or H₂C═C(R⁵)—(C═O)—O—(—CH₂—CH(R⁸)—O—)ₖ—R^{7a} (IIId).

In the formulae (IIc) and (IId), R⁵ is as defined above, and the —O—(—CH₂—CH(R⁸)—O—)ₖ— and —(C═O)—O—(—CH₂—CH(R⁸)—O—)ₖ— groups are each specific linking R⁶ groups, meaning that (IIc) is a vinyl ether and (IId) is an acrylic ester.

The number of alkylene oxide units k is a number from 10 to 80, preferably 12 to 60, more preferably 15 to 50 and, for example, 20 to 40. It will be apparent to the person skilled in the art in the field of alkylene oxides that the values stated are mean values.

The R⁸ radicals are each independently H, methyl or ethyl, preferably H or methyl, with the proviso that at least 70 mol % of the R⁸ radicals are H. Preferably at least 80 mol % of the R⁸ radicals are H, more preferably at least 90 mol %, and they are most preferably exclusively H. The block mentioned is thus a polyoxyethylene block which may optionally also have certain proportions of propylene oxide and/or butylene oxide units, preferably a pure polyoxyethylene block.

R^{7a} is an aliphatic and/or aromatic, straight-chain or branched hydrocarbyl radical having 8 to 40 carbon atoms, preferably 12 to 32 carbon atoms. In one embodiment, the aliphatic hydrocarbyl groups have 8 to 22, preferably 12 to 18 carbon atoms. Examples of such groups include n-octyl, n-decyl, n-dodecyl, n-tetradecyl, n-hexadecyl or n-octadecyl groups. In a further embodiment, the groups are aromatic groups, especially substituted phenyl radicals, especially distyrylphenyl groups and/or tristyrylphenyl groups.

In a further embodiment of the invention, the monomers (A2) are monomers of the general formula

H₂C═C(R⁵)—R⁹—O—(—CH₂—CH(R¹⁰)—O—)ₓ—(—CH₂—CH(R¹¹)—O—)_{y}—(—CH₂—CH₂O—)_{z}—R¹² (IIe).

In the monomers (A2) of the formula (IIe), an ethylenic group H₂C═C(R⁵)— is bonded via a divalent, linking group —R⁹—O— to a polyoxyalkylene radical having block structure, where the —CH₂—CH(R¹⁰)—O—)ₓ—, —(—CH₂—CH(R¹¹)—O—)^{l}— and optionally —CH₂—CH₂O—)_{z}—R¹² blocks are arranged in the order shown in formula (IIe). The transition between the two blocks may be abrupt or else continuous.

In formula (IIe), R⁵ is as already defined, i.e. R⁵ is H or a methyl group.

R⁹ is a single bond or a divalent linking group selected from the group consisting of —(CₙH₂ₙ)—[R^{9a} group], —O—(C_{n′}H_{2n′})— [R^{9b} group]— and —C(O)—O—(C_{n″}H_{2n′}′)— [R^{9c} group]. In the formulae stated, each n is a natural number from 1 to 6, n′ and n″ are each a natural number from 2 to 6. In other words, the linking group comprises straight-chain or branched aliphatic hydrocarbyl groups having 1 to 6 hydrocarbon atoms, which are bonded to the ethylenic group H₂C═C(R⁵)— directly, via an ether group —O— or via an ester group —C(O)—O—. Preferably, the —(CₙH₂ₙ)—, —(C_{n′}H_{2n′}) and —(C_{n″}H_{2n″})— groups are linear aliphatic hydrocarbyl groups.

Preferably, the R^{9a} group is a group selected from 1'CH₂—, —CH₂—CH₂— and —CH₂—CH₂—CH₂—, particular preference being given to a methylene group —CH₂—.

Preferably, the R^{9b} group is a group selected from —O—CH₂—CH₂—, —O—CH₂—CH₂—CH₂— and —O—CH₂—CH₂—CH₂—CH₂—, more preferably —O—CH₂—CH₂—CH₂—CH₂—.

Preferably, the R^{9c} group is a group selected from —C(O)—O—CH₂—CH₂—, —C(O)—O—CH(CH₃)—CH₂—, —C(O)O—CH₂—CH(CH₃)—, —C(O)O—CH₂—CH₂—CH₂—CH₂— and —C(O)O—CH₂—CH₂—CH₂—CH₂—CH₂—CH₂—, more preferably —C(O)—O—CH₂—CH₂— and —C(O)O—CH₂—CH₂—CH₂—CH₂— and most preferably —C(O)—O—CH₂—CH₂—.

More preferably, the R⁹ group is an R^{9b} group, most preferably —O—CH₂—CH₂—CH₂—CH₂—.

In the —(—CH₂—CH(R¹⁰)—O—)ₓ block, the R¹⁰ radicals are each independently H, methyl or ethyl, preferably H or methyl, with the proviso that at least 70 mol % of the R¹⁰ radicals are H. Preferably at least 80 mol % of the R¹⁰ radicals are H, more preferably at least 90 mol %, and they are most preferably exclusively H. The block mentioned is thus a polyoxyethylene block which may optionally have certain proportions of propylene oxide and/or butylene oxide units, preferably a pure polyoxyethylene block.

The number of alkylene oxide units x is a number from 10 to 50, preferably 12 to 40, more preferably 15 to 35, even more preferably 20 to 30 and is, for example, about 22 to 25. It will be apparent to the person skilled in the art in the field of polyalkylene oxides that the numbers stated are mean values of distributions.

In the second —(—CH₂—CH(R¹¹)—O—)_{y}— block, the R¹¹ radicals are each independently hydrocarbyl radicals of at least 2 carbon atoms, for example 2 to 10 carbon atoms, preferably 2 or 3 carbon atoms. This radical may be an aliphatic and/or aromatic, linear or branched carbon radical. Preference is given to aliphatic radicals.

Examples of suitable R¹¹ radicals include ethyl, n-propyl, n-butyl, n-pentyl, n-hexyl, n-heptyl, n-octyl, n-nonyl or n-decyl, and phenyl. Examples of preferred radicals include ethyl, n-propyl, n-butyl, n-pentyl, and particular preference is given to ethyl and/or n-propyl radicals. The —(—CH₂—CH(R¹¹)—O—)_{y}— block is thus a block consisting of alkylene oxide units having at least 4 carbon atoms.

The number of alkylene oxide units y is a number from 5 to 30, preferably 8 to 25.

In formula (IIe), z is a number from 0 to 5, for example 1 to 4, i.e. the terminal block of ethylene oxide units is thus merely optionally present. In a preferred embodiment of the invention, it is possible to use a mixture of at least two monomers (A2) of the formula (IIe), where the R⁵, R⁹,

R¹⁰, R¹¹, R¹² radicals and indices x and y are each the same, but in one of the monomers z=0 while z>0 in the other, preferably 1 to 4.

The R¹² radical is H or a preferably aliphatic hydrocarbyl radical having 1 to 30 carbon atoms, preferably 1 to 10 and more preferably 1 to 5 carbon atoms. Preferably, R¹² is H, methyl or ethyl, more preferably H or methyl and most preferably H.

The hydrophobically associating monomers (A2) of the formulae (IIc), (IId) and (IIe), acrylamide copolymers comprising these monomers and the preparation thereof are known in principle to those skilled in the art, for example from WO 2010/133527 and WO 2012/069478.

In a further embodiment, the associative monomer (A2) is a cationic monomer of the general formula H₂C═C(R⁵)—C(═O)O—R¹³—N⁺(R¹⁴)(R¹⁵)(R¹⁶) X⁻ (IIf) or H₂C═C(R⁵)—C(═O)N(R¹⁷)—R¹³—N⁺(R¹⁴)(R¹⁵)(R¹⁶) X⁻ (IIg).

In the formulae (IIf) and (IIg), R⁵ is as defined above.

R¹³ is an alkylene radical, especially a ω-alkylene radical having 1 to 8 carbon atoms, preferably 2 to 4 carbon atoms and especially 2 or 3 carbon atoms. Examples include —CH₂—, —CH₂CH₂—, —CH₂CH₂CH₂— and —CH₂CH₂CH₂CH₂—. Particular preference is given to —CH₂CH₂— and —CH₂CH₂CH₂—.

R¹³, R¹⁴ and R¹⁵ are each independently H or an alkyl group having 1 to 4 carbon atoms, preferably H or methyl. R¹³ is preferably H, and R¹⁴ and R¹⁵ are preferably each methyl. X⁻ is a negatively charged counterion, especially a halide ion selected from F⁻, Cl⁻, Br and I⁻, preferably Cl⁻ and/or Br.

R¹⁶ is an aliphatic and/or aromatic, linear or branched hydrocarbyl group having 8 to 30 carbon atoms, preferably 12 to 18 carbon atoms. R¹⁶ may especially comprise aliphatic hydrocarbyl radicals having 8 to 18, preferably 12 to 18 carbon atoms. Examples of such groups include n-octyl, n-decyl, n-dodecyl, n-tetradecyl, n-hexadecyl or n-octadecyl groups, preference being given to n-dodecyl, n-tetradecyl, n-hexadecyl or n-octadecyl groups.

Preference is given to a monomer of the general formula (IIg). Examples of such monomers include N-(meth)acrylamidopropyl-N,N-dimethyl-N-dodecylammonium chloride, N-(meth)acrylamidopropyl-N,N-dimethyl-N-tetradecylammonium chloride, N-(meth)acrylamidopropyl-N,N-dimethyl-N-hexadecylammonium chloride or N-(meth)acrylamidopropyl-N,N-dimethyl-N-octadecylammonium chloride or the corresponding bromides. Monomers of this kind, and acrylamide copolymers having monomers of this kind, are known and described, for example, in U.S. Pat. No. 7,700,702 B2.

As well as the hydrophilic monomers (A1) and/or associative monomers (A2), acrylamide copolymers may optionally comprise ethylenically unsaturated monomers other than the monomers (A1) and (A2), preferably monoethylenically unsaturated monomers (A3). It is of course also possible to use mixtures of various monomers (A3). Monomers of this kind can be used for fine control of the properties of acrylamide copolymers.

The monomers (A3) may, for example, be monoethylenically unsaturated monomers which have a more hydrophobic character than the hydrophilic monomers (A1) and which are correspondingly water-soluble only to a small degree. In general, the solubility of the monomers (A3) in water at room temperature is less than 50 g/l, especially less than 30 g/l. Examples of monomers of this kind include N-alkyl- and N,N′-dialkyl(meth)acrylamides, where the number of carbon atoms in the alkyl radicals together is at least 3, preferably at least 4. Examples of monomers of this kind include N-butyl(meth)acrylamide, N-cyclohexyl(meth)acrylamide and N-benzyl(meth)acrylamide.

In addition, monomers (A3) may also be ethylenically unsaturated monomers having more than one ethylenic group. Monomers of this kind can be used in special cases in order to achieve easy crosslinking of the acrylamide polymers. The amount thereof should generally not exceed 2% by weight, preferably 1% by weight and especially 0.5% by weight, based on the sum total of all the monomers. More preferably, the monomers (A3) are exclusively monoethylenically unsaturated monomers.

One embodiment of the invention involves a homopolymer of methacrylamide or of acrylamide, preferably a homopolymer of acrylamide. The term “homopolymer” shall also include copolymers of acrylamide and methacrylamide

(Meth)acrylamide copolymers comprise, as well as (meth)acrylamide, preferably acrylamide, at least one further, monoethylenically unsaturated monomer other than (meth)acrylamide. This is at least one monomer selected from the group of non-(meth)acrylamide hydrophilic monomers (A1), amphiphilic monomers (A2) or further monomers (A3). Preferred (meth)acrylamide copolymers comprise, as well as (meth)acrylamide, at least one further, different hydrophilic monomer (A1). Other preferred (meth)acrylamide copolymers comprise, as well as (meth)acrylamide, at least one further, different hydrophilic monomer (A1) and at least one hydrophilic monomer (A2).

The amount of all the hydrophilic monomers (A1) together, i.e. including (meth)acrylamide, is at least 70% by weight based on the amount of all the monomers, preferably at least 80% by weight and more preferably at least 90% by weight.

In (meth)acrylamide copolymers, generally at least 20% by weight, especially at least 30% by weight, preferably at least 50% by weight, more preferably at least 60% by weight and, for example, at least 70% by weight of the monoethylenically unsaturated monomers (A) are (meth)acrylamide, where the stated amount is based on the sum total of all the monomers.

If present, the amount of amphiphilic monomers (A2) may be up to 15% by weight, based on the total amount of all the monomers in acrylamide copolymers, for example 0.1 to 15% by weight, especially 0.2 to 10% by weight, preferably 0.5 to 5% by weight and, for example, 0.5 to 2% by weight.

If they are present at all, the amount of optionally present monomers (A3) may be up to 15% by weight, preferably up to 10% by weight, more preferably up to 5% by weight, based in each case on the total amount of all the monomers. An upper limit for ethylenically unsaturated monomers having more than one ethylenic group has already been given. Most preferably, no monomers (A3) are present.

Apart from the monomers (A1), (A2) and (A3), it is generally the case that no further monomers are present, i.e. the sum total of the monomers (A1), (A2) and (A3) is generally 100%.

In one embodiment of the invention, the copolymer is a copolymer comprising 85% by weight to 99.9% by weight of hydrophilic monomers (A1) including at least (meth)acrylamide, preferably 90% by weight to 99.8% by weight, more preferably 95% by weight to 99.5, and 0.1% by weight to 15% by weight of amphiphilic monomers (A2), preferably 0.2% by weight to 10% by weight, more preferably 0.5% by weight to 5% by weight, where the sum of all the monomers (A1) and (A2) is 100% by weight.

In a preferred embodiment, the (meth)acrylamide polymer is a copolymer comprising (meth)acrylamide and at least one anionic, monoethylenically unsaturated, hydrophilic monomer (A1b). More particularly, the monomer (A1b) is a monomer comprising at least one acidic group selected from the group of —COOH, —SO₃H or —PO₃H₂ or salts thereof, preferably —COOH and/or —SO₃H or salts thereof.

In a preferred embodiment, the acrylamide polymer is a copolymer comprising (meth)acrylamide and acrylic acid or salts thereof. This may especially be a copolymer comprising 60 to 80% by weight of (meth)acrylamide and 20 to 40% by weight of acrylic acid. Optionally, the copolymer may comprise at least one amphiphilic copolymer (A2) in an amount of up to 15% by weight, preferably 0.2 to 10% by weight. More preferably, this is an amphiphilic monomer of the general formula (IIe) H₂C═C(R⁵)—R⁹—O—(—CH₂—CH(R¹⁰)—O—)ₓ—(—CH₂—CH(R¹¹)—O—)_{y}—(—CH₂—CH₂O—)_{z}—R¹². The radicals and indices and the preferred ranges thereof have already been defined above.

In a further preferred embodiment, the acrylamide polymer is a copolymer comprising (meth)acrylamide and ATBS (2-acrylamido-2-methylpropane-1-sulfonic acid, H₂C═CH—CO—NH—C(CH₃)₂—CH₂—SO₃H or salts thereof. This may especially be a copolymer comprising 40 to 60% by weight of (meth)acrylamide and 40 to 60% by weight of AMPS. Optionally, the copolymer may comprise at least one amphiphilic comonomer (A2) in an amount of up to 15% by weight, preferably 0.2 to 10% by weight. More preferably, this is an amphiphilic monomer of the general formula (IIe) H₂C═C(R⁵)—R⁹—O—(—CH₂—CH(R₁₀)—O—)ₓ—(—CH₂—CH(R¹¹)—O—)_{y}—(—CH₂—CH₂—O—)_{z}—R¹². The radicals and indices and the preferred ranges thereof have already been defined above.

In a further preferred embodiment, the (meth)acrylamide polymer is a copolymer comprising (meth)acrylamide and at least two anionic, monoethylenically unsaturated, hydrophilic monomers (A1b).

More particularly, the monomers (A1b) are monomers comprising at least one acidic group selected from the group of —COOH, —SO₃H or —PO₃H₂ or salts thereof, preferably —COOH and/or —SO₃H or salts thereof. An acrylamide polymer of this kind is preferably a copolymer comprising (meth)acrylamide, 2-acrylamido-2-methylpropanesulfonic acid (AMPS) and acrylic acid. This may especially be a copolymer comprising 40 to 60% by weight of (meth)acrylamide and 20 to 30% by weight of acrylic acid and 20 to 30% by weight of AMPS. Optionally, the copolymer may comprise at least one amphiphilic comonomer (A2) in an amount of up to 15% by weight, preferably 0.2 to 10% by weight. More preferably, this is an amphiphilic monomer of the general formula (IIe) H₂C═C(R⁵)—R⁹—O—(—CH₂—CH(R¹⁰)—O—)ₓ—(—CH₂—CH(R¹¹)—O—)_{y}—(—CH₂—CH₂O—)_{z}—R¹². The radicals and indices and the preferred ranges thereof have already been defined.

In a further preferred embodiment, the (meth)acrylamide polymer is a copolymer comprising (meth)acrylamide and at least one cationic, monoethylenically unsaturated, hydrophilic monomer (A1c). The monomers (A1c) may especially be monomers H₂C═C(R¹)—CO—NR²—R³—N(R⁴)₃+X⁻ (Ia) and/or H₂C═C(R¹)—COO—R³—N(R⁴)₃⁺ X⁻ (Ib). The radicals and indices and the preferred ranges thereof have already been defined above. This may especially be a copolymer comprising 60 to 80% by weight of (meth)acrylamide and 20 to 40% by weight of cationic monomers (A1c). Optionally, the copolymer may comprise at least one amphiphilic comonomer (A2) in an amount of up to 15% by weight, preferably 0.2 to 10% by weight.

In a further preferred embodiment, the (meth)acrylamide polymer is a copolymer comprising (meth)acrylamide, at least one anionic, monoethylenically unsaturated, hydrophilic monomer (A1b) and at least one amphiphilic monomer (A2) of the general formula H₂C═C(R⁵)—C(═O)O—R¹³—N⁺(R¹⁴)(R¹⁵)(R¹⁶) X⁻ (IIf) or H₂C═C(R⁵)—C(═O)N(R¹⁷)—R¹³—N⁺ (R¹⁴)(R¹⁵)(R¹⁶) X⁻ (IIg). It is preferably a monomer of the general formula (IIg). The radicals and indices and the preferred ranges thereof have already been defined above. This may especially be a copolymer comprising 60 to 80% by weight of (meth)acrylamide and 10 to 40% by weight of anionic monomers (A1b) and 0.1 to 10% by weight of said monomer (A2) of the formula (IIf) and/or (IIg), preferably (IIg).

### Use of the Aqueous Poly Acrylamide Solutions

The aqueous polyacrylamide solutions manufactured according to the present invention may be used for various purposes, for example for mining applications, oilfield applications, including but not limited to the application in enhanced oil recovery, oil well drilling or as friction reducer, or waste water cleanup, water treatment, paper making or agricultural applications. The composition of the polyacrylamide solutions is selected by the skilled artisan according to the intended use of the polyacrylamide solution.

### Enhanced Oil Recovery

In one embodiment of the invention, the method for manufacturing aqueous polyacrylamide solutions according to the present invention is carried out on an oilfield and the polyacrylamide solution thus manufactured is used for enhanced oil recovery.

Accordingly, the present invention also relates the use of aqueous polyacrylamide solutions for producing mineral oil from underground mineral oil deposits by injecting an aqueous fluid comprising at least an aqueous poly acrylamide solution into a mineral oil deposit through at least one injection well and withdrawing crude oil from the deposit through at least one production well, wherein the aqueous polyacrylamide solution is prepared on the oilfield using a process comprising the following steps, particularly in the given order:
hydrating acrylonitrile in water in presence of a biocatalyst capable of converting acrylonitrile to acrylamide so as to obtain an acrylamide solution,
directly polymerizing the acrylamide solution so as to obtain a polyacrylamide gel, and
directly dissolving the polyacrylamide gel by addition of water so as to obtain an aqueous polyacrylamide solution.

For the inventive use, at least one production well and at least one injection well are sunk into the mineral oil deposit. In general, a deposit will be provided with a plurality of injection wells and with a plurality of production wells. An aqueous fluid is injected into the mineral oil deposit through the at least one injection well, and mineral oil is withdrawn from the deposit through at least one production well. By virtue of the pressure generated by the aqueous fluid injected, called the “polymer flood”, the mineral oil flows in the direction of the production well and is produced through the production well. In this context, the term “mineral oil” does not of course just mean a single-phase oil; instead, the term also encompasses the customary crude oil-water emulsions.

The aqueous fluid for injection comprises the aqueous poly acrylamide solution prepared according to the process according to the present invention. Details of the process have been disclosed above. The aqueous acryl amide solution obtained may be used as such or it may be mixed with further components. Further components for enhanced oil recovery fluids may be selected by the skilled artisan according to his/her needs.

For enhanced oil recovery, a homopolymer of acryl amide may be used, however preferably copolymers of acryl amide and one or more additional monoethylenically unsaturated, hydrophilic monomers are used.

In one embodiment, the acryl amide copolymers comprise at least one hydrophilic, anionic monomer (A1b) comprising at least one acidic group, or salts thereof. Examples of such monomers (A1b) have been disclosed above.

Preferably, monomer (A1b) may be selected from the group consisting of acrylic acid, methacrylic acid, crotonic acid, itaconic acid, maleic acid, fumaric acid, vinylsulfonic acid, allylsulfonic acid, 2-acrylamido-2-methylpropanesulfonic acid (AMPS), 2-methacrylamido-2-methylpropane-sulfonic acid, 2-acrylamidobutanesulfonic acid, 3-acrylamido-3-methylbutane-sulfonic acid, 2-acrylamido-2,4,4-trimethylpentanesulfonic acid, vinylphosphonic acid, allylphosphonic acid, N-(meth)acrylamidoalkylphosphonic acids and (meth)acryloyloxyalkyl-phosphonic acids, more preferably from acrylic acid and/or APMS or salts thereof.

In such copolymers comprising acryl amide and monomers (A1b), preferably acrylic acid and/or APMS or salts thereof, the amount of acryl amide usually is from 40% by wt. to 90% by wt. and the amount of monomers (A1b) is from 10% by wt. to 60% by wt., relating to the amount of all monomers in the copolymer. Preferably, the amount of acryl amide is from 60% by wt. to 80% by wt. and the amount of monomers (A1b) is from 20% by wt. to 40% by wt.,

In another embodiment, the acryl amide copolymers comprise at least one hydrophilic, anionic monomer (A1b) comprising at least one acidic group, or salts thereof, preferably acrylic acid and/or APMS or salts thereof, and at least one amphiphilic monomer (A2). Examples of amphiphilic monomers (A2) have been disclosed above.

Preferably, the monomers (A2) are monomers of the general formula H₂C═C(R⁵)—R⁹—O—(—CH₂—CH(R¹⁰)—O—)ₓ—(—CH₂—CH(R¹¹)—O—)_{y}—(—CH₂—CH₂—)_{z}—R¹² (IIe). The definitions of R⁵, R⁹, R¹⁰, R¹¹, R¹² and x, y, z in (IIe) have been disclosed above and we refer to said definitions, including the preferred embodiments.

The amount of amphiphilic monomers (A2), in particular those of formula (IIe) may be up to 15% by weight, based on the total amount of all the monomers in acrylamide copolymers, for example 0.1 to 15% by weight, especially 0.2 to 10% by weight, preferably 0.5 to 5% by weight and, for example, 0.5 to 2% by weight.

In such copolymers comprising acryl amide, monomers (A1b), preferably acrylic acid and/or APMS or salts thereof, and monomers (A2), preferably of formula (IIe), usually the amount of acryl amide is from 40% by wt. to 89.9% by wt., the amount of monomers (A1b) is from 10% by wt. to 59.9% by wt., and the amount of amphiphilic monomers (A2) is from 0.1 to 15% by wt. relating to the amount of all monomers in the copolymer. Preferably, the amount of acryl amide is from 40% by wt. to 59.5% by wt., the amount of monomers (A1b) is from 40% by wt. to 59.5% by wt., and the amount of amphiphilic monomers (A2) is from 0.5 to 2% by wt.

The aqueous fluid for injection can be made up in freshwater or else in water comprising salts, such as seawater or formation water. Water comprising salts may already be used for dissolving the polyacrylamide gel. Alternatively, the polyacrylamide gel may be dissolved in fresh water, and the solution obtained can be diluted to the desired use concentration with water comprising salts.

The aqueous injection fluid may of course optionally comprise further components. Examples of further components include biocides, stabilizers, free-radical scavengers, initiators, surfactants, cosolvents, bases and complexing agents.

The concentration of the copolymer in the injection fluid is fixed such that the aqueous formulation has the desired viscosity for the end use. The viscosity of the formulation should generally be at least 5 mPas (measured at 25° C. and a shear rate of 7 s⁻¹), preferably at least 10 mPas.

In general, the concentration of the polyacrylamide in the injection fluid is 0.02 to 2% by weight based on the sum total of all the components in the aqueous formulation. The amount is preferably 0.05 to 0.5% by weight, more preferably 0.1 to 0.3% by weight and, for example, 0.1 to 0.2% by weight.

### Mining Applications

In one embodiment, the method for preparing an aqueous polyacrylamide solution according to the present invention is carried out in areas where mining, mineral processing and/or metallurgy activities takes place. Consequently, the aqueous polyacrylamide solution as product obtained by the method of the present invention is preferably used for applications in the field of mining, mineral processing and/or metallurgy and the method for preparing the aqueous polyacrylamide solution is preferably used at the plant of the respective industry.

Preferably, mining activities comprises extraction of valuable minerals or other geological materials from certain deposits. Such deposits can contain ores, for example metal containing ores, sulfidic ores and/or non-sulfidic ores. The ores may comprise metals, coal, gemstones, limestone or other mineral material. Mining is generally required to obtain any material in particular mineral material that cannot be grown through agricultural processes, or created artificially in a laboratory or factory. The aqueous polyacrylamide solution according to the present invention is preferably used to facilitate the recovery of mineral material, for beneficiation of ores and for further processing of ores to obtain the desired minerals or metals.

Typically, mining industries, mineral processing industries and/or metallurgy industries are active in the processing of ores and in the production of for example alumina, coal, iron, steel, base metals, precious metals, diamonds, non-metallic minerals and/or areas where aggregates play an important role. In such industries, the method of the present invention and the obtained homo- or copolymer of acrylamide can be used for example
at plants for alumina production, where alumina is extracted from the mineral bauxite using the Bayer caustic leach process,
at plants where the coal washing process demands a closed water circuit and efficient tailings disposal to satisfy both economic and environmental demands,
at plants for iron and steel production, where the agglomeration of fine iron concentrates to produce pellets of high quality is a major challenge for the iron ore industry,
at plants for base metal production, where flocculants find several uses in base metal production,
at plants for precious metals production, where reagents are used to enhance the tailings clarification process allowing the reuse of clean water,
at diamond plants, where efficient water recovery is paramount in the arid areas where diamonds are often found,
at plants for non-metallic mineral production where reagents enhance water recovery or aid the filtration processes to maximize process efficiency,
at plants where aggregates have to be produced and flocculants and filter aids are needed to enhance solid/liquid separation.

Accordingly, the present invention relates to the use of an aqueous polyacrylamide solution for mining, mineral processing and/or metallurgy activities comprising the use for solid liquid separation, for tailings disposal, for polymer modified tailings deposition, for tailings management, as density and/or rheology modifier, as agglomeration aid, as binder and/or for material handling, wherein the aqueous polyacrylamide solution is prepared at the plant of the respective industry, comprising the following steps in the given order:
hydrating acrylonitrile in water in presence of a biocatalyst capable of converting acrylonitrile to acrylamide so as to obtain an acrylamide solution,
directly polymerizing the acrylamide solution so as to obtain a polyacrylamide gel, and
directly dissolving the polyacrylamide gel by addition of water so as to obtain an aqueous polyacrylamide solution.

For the mining, mineral processing and/or metallurgy activities a homopolymer of acrylamide for example can be used. Further preferred are also copolymers of acrylamide. Such copolymers of acrylamide can be anionic, cationic or non-ionic. Anionic copolymers are for example copolymers of acrylamide with increasing proportions of acrylate groups, which give the polymers negative charges, and thus anionic active character, in aqueous solution. Anionic copolymers of acrylamide can in particular be used for waste water treatment in metallurgy like iron ore plants, steel plants, plants for electroplating, for coal washing or as flocculants. Non-ionic polymers and/or copolymers of acrylamide can be used for example as nonionic flocculants suitable as settlement aids in many different mineral processing applications and are particularly effective under very low pH conditions, as encountered for example in acidic leach operations. Cationic copolymers of acrylamide have in particular an increasing proportion of cationic monomers. The cationic groups, which are thus introduced into the polymer, have positive charges in aqueous solution.

It is preferred, that the polymer obtained from the method of the present invention is used as flocculant in a process in which individual particles of a suspension form aggregates. The polymeric materials of the present invention forms for example bridges between individual particles in the way that segments of the polymer chain adsorb on different particles and help particles to aggregate. Consequently, the polymers of the present invention act as agglomeration aid, which may be a flocculant that carries active groups with a charge and which may counterbalance the charge of the individual particles of a suspension. The polymeric flocculant may also adsorb on particles and may cause destabilization either by bridging or by charge neutralization. In case the polymer is an anionic flocculant, it may react against a positively charged suspension (positive zeta potential) in presence of salts and metallic hydroxides as suspension particles, for example. In case the polymer of the present invention is for example a cationic flocculant, it may react against a negatively charged suspension (negative zeta potential) like in presence of for example silica or organic substances as suspension particles. For example, the polymer obtained from the method of the present invention may be an anionic flocculant that agglomerates clays which are electronegative.

Preferably, the method of the present invention and the obtained polymer and/or copolymer of acrylamide (polyacrylamide) is used for example in the Bayer process for alumina production. In particular, the polyacrylamide can be used as flocculant in the first step of the Bayer-Process, where the aluminum ore (bauxite) is washed with NaOH and soluble sodium aluminate as well as red mud is obtained. Advantageously, the flocculation of red mud is enhanced and a faster settling rate is achieved when acrylamide polymers and/or co-polymers are added. As red mud setting flocculants, polyacrylamide may be used for settling aluminum red mud slurries in alumina plants, provides high settling rates, offers better separation performance and reduces suspended solids significantly. Also the liquor filtration operations are improved and with that the processing is made economically more efficient. It is further preferred that the polyacrylamides are used in decanters, in washers, for hydrate thickening, for green liquor filtration, as crystal growth modifiers, as thickener and/or as rheology modifier.

It is further preferred that the method of the present invention and the polymers of acrylamide are used in processes for solid liquid separation as for example flocculant or dewatering aid, which facilitate thickening, clarifying, filtration and centrifugation in order to enhance settling rates, to improve clarities and to reduce underflow volumes. In particular, in filtration processes the polyacrylamide homo- or co-polymer of the present invention increase filtration rates and yields, as well as reducing cake moisture contents.

Further preferred is the use of the method and the obtained polyacrylamide of the present invention in particular for material handling and as binder. In the mining industry, the movement of large volumes of material is required for processing the rock and/or ores which have been extracted from the deposits. The typical rock and/or ore processing for example starts with ore extraction, followed by crushing and grinding the ore, subsequent mineral processing (processing or the desired/valuable mineral material), then for example metal production and finally the disposal of waste material or tailings. It was a surprise that with the method of the present invention and in particular the obtained polyacrylamide the handling of the mineral material can be enhanced by increasing efficiency and yield, by improving product quality and by minimizing operating costs. Particularly, the present invention can be used for a safer working environment at the mine site and for reduction of environmental discharges.

Preferably, the method and the obtained polyacrylamide of the present invention can for example be used as thickener, as density and/or rheology modifier, for tailings management. The obtained polyacrylamide polymer can modify the behavior of the tailings for example by rheological adjustment. The obtained polyacrylamide polymers are able to rigidify tailings at the point of disposal by initiating instantaneous water release from the treated slurry. This accelerates the drying time of the tailings, results in a smaller tailings footprint and allows the released water to be returned to the process faster. This treatment is effective in improving tailings properties in industries producing alumina, nickel, gold, iron ore, mineral sands, oil sands or copper for example. Further benefits of the polymers obtained according to the present invention are for example maximized life of disposal area, slurry placement control, no re-working of deposit required, co-disposal of coarse and fine material, faster trafficable surface, reduced evaporative losses, increased volume for recycling, removed fines contamination, reduced fresh water requirement, lower land management cost, less mobile equipment, lower rehabilitation costs, quicker rehabilitation time, lower energy consumption, accelerated and increased overall water release, improved rate of consolidation, reduced rate of rise, reduced amount of post depositional settlement.

Preferably, the obtained product from the method of the present invention is used for agglomeration of fine particulate matter and for the suppression of dust. Particularly, polyacrylamide polymers or copolymers are used as organic binders to agglomerate a wide variety of mineral substrates. For example, the polyacrylamide polymers or copolymers are used for iron ore pelletization as a full or partial replacement for bentonite. The product from the method of the present invention can be used as binder, in particular as solid and liquid organic binders in briquetting, extrusion, pelletization, spheronization and/or granulation applications and gives for example excellent lubrication, molding and/or binding properties for processes such as coal-fines briquetting, carbon extrusion, graphite extrusion and/or nickel briquetting.

It is preferred that the method of the present invention and in particular the aqueous polyacrylamide solution obtained by the method is used for the beneficiation of ores which comprise for example coal, copper, alumina, gold, silver, lead, zinc, phosphate, potassium, nickel, iron, manganese, or other minerals.

The method according to the present invention will be described in further detail based on the following example.

### EXAMPLE 1

The method is carried out on site. Particularly, the method is carried out in at least one mobile reactor. For example, the installation 10 is provided on a vehicle. The first reactor 12 is supplied with 1,554.18 g acrylonitrile, 2,609.24 g water and 1.67 g biocatalyst capable of converting acrylonitrile to acrylamide. The biocatalyst is rhodococcus rhodochrous. The biocatalyst is provided as a powder. Within the first reactor 12, the acrylonitrile is hydrated in water in presence of the biocatalyst so as to obtain an acrylamide solution. The hydrating is carried out at ambient temperature, i.e. 25° C., and atmospheric pressure. The hydrating takes 7 h. Thereby, the acrylamide solution comprises a concentration of 50% by weight acrylamide monomers. The thus obtained acrylamide solution is directly and immediately after its preparation supplied to the second reactor 14, wherein the biocatalyst is removed, e.g. by means of the filter within the pipe 18.

The acrylamide solution is cooled to a temperature of 4° C. before entering the second reactor 14. For this purpose, a heat exchanger is present within the pipe 18. The second reactor 14 is not only supplied with the acrylamide solution but also with 2,622.9 g of sodium acrylate solution (35% in water), 2,966 g of water, 50 g of a suspension of azobisisobutyronitrile (AlBN) in water (4% active content) and 75 g of a solution 4,4′-Azobis(4-cyanovaleric acid) (ACVA) in 1N NaOH solution (4% active content of ACVA) and a redox initiator system comprising tBHP and sodium sulfite, which is added to the acrylamide solution for initiating a polymerization process. The redox initiator is added with a concentration of 1% by weight in water and a final concentration of the redox initiators is set to 2.4 ppm for sodium sulfite and 4.8 ppm for tBHP (on the whole reaction mixture). Thus, the acrylamide solution is directly polymerized so as to obtain a polyacrylamide gel. The polymerization is carried out at atmospheric pressure. The polyacrylamide gel comprises 30% polyacrylamide solids (by means of a copolymer comprising approx. 75 mol % of acrylamide). The polymerization takes 7 h.

Thus, approx. 10 kg polyacrylamide gel is obtained. The thus obtained polyacrylamide gel is directly and immediately after its preparation supplied to the static mixer 16. The static mixers 16 are from Fluitec mixing+reaction solutions AG, Seuzachstrasse 40, 8413 Neftenbach, Switzerland. Particularly, a first sub tube having an inner diameter of 36 mm, a length of 1,000 mm and equipped with a static mixer of the type CSE-W was used. A second sub tube having an inner diameter of 36 mm, a length 270 mm and equipped with a static mixer of the type CSE-W was used. Further, a third sub tube having an inner diameter 36 mm, a length of 1,000 mm and equipped with a static mixer of the type CSE-X was used. The sub tubes were connected in series to form a U-Shape, wherein the first sub tube and the third sub tube are parallel to each other and are perpendicular to the second sub tube. The polyacrylamide gel is supplied with kg/h. Water is added to the polyacrylamide gel with 25 kg/h for dissolving the same by means of the static mixer 16 so as to obtain an aqueous polyacrylamide solution. The polyacrylamide gel is dissolved with a resting time within the mixers 16 of 0.05 s to 10 s and preferably 0.1 s to 2 s. The resulting suspension of gel particles with a size of 1-2 mm was subsequently diluted with 272 kg water to obtain an active content of 1% by weight of polyacrylamide. This suspension turned into a discrete solution within 1 h under slow stirring. The intrinsic viscosity of the solution was 24 dl/g.

Thereby, the aqueous polyacrylamide solution is prepared so as to be suitable in oil recovery and /or mining. According to the times described before, the complete method is carried out in a time of 15 h. The method is monitored on line by means of a plurality of sensors provided within the pipes 18, 20 and the reactors 12, 14.

1-21. (canceled)

22. A method for preparing an aqueous polyacrylamide solution, the method comprising:
hydrating acrylonitrile in water in the presence of a biocatalyst, to obtain an acrylamide solution, wherein the biocatalyst is capable of converting acrylonitrile to acrylamide,
directly polymerizing the acrylamide solution, to obtain a polyacrylamide gel, wherein the polyacrylamide gel comprises 16 to 50% by weight of polyacrylamide solids, and
directly dissolving the polyacrylamide gel by adding water, to obtain an aqueous polyacrylamide solution,
wherein the method is carried out on site.

23. The method of claim 22, wherein the polyacrylamide gel is dissolved with at least one static mixer.

24. The method of claim 22, wherein the aqueous polyacrylamide solution comprises 0.03 to 5.0% by weight of polyacrylamide.

25. The method of claim 23, wherein the polyacrylamide gel is dissolved with a resting time within the at least one static mixer of 0.05 to 10 s.

26. The method of claim 22, wherein the biocatalyst encodes the enzyme nitrile hydratase.

27. The method of claim 22, wherein the biocatalyst is a nitrile hydratase producing microorganism.

28. The method of claim 22, further comprising:
adding at least one monoethylenically unsaturated, water-soluble comonomer to the acrylamide solution.

29. The method of claim 28, wherein the at least one monoethylenically unsaturated, water-soluble comonomer is selected from the group consisting of acrylic acid, 2-acrylamido-2-methylpropane sulfonic acid and a salt thereof.

30. The method of claim 29, wherein an amount of the at least one monoethylenically unsaturated, water-soluble comonomer is 25 to 40% by weight relative to a total amount of all monomers.

31. The method of claim 22, wherein the biocatalyst is removed before directly polymerizing the acrylamide solution.

32. The method of claim 22, wherein a conversion of acrylonitrile to acrylamide is carried out at a starting temperature of 15 to 30° C.

33. The method of claim 22, wherein the polymerization of the acrylamide is initiated by adding an initiator for radical polymerization.

34. The method of claim 33, wherein the initiator is selected from the group consisting of a peroxide, a persulfate, an azo compound, a redox couple and a mixture thereof.

35. The method of claim 22, wherein the method is monitored on line.

36. The method of claim 22, wherein the method is carried out at an oilfield or a mining area.

37. The method of claim 22, wherein the method is carried out in at least one mobile reactor.

38. The method of claim 22, wherein the method is carried out for 12 to 72 h.

39. A process for producing mineral oil from an underground mineral oil deposit, the process comprising:
a) preparing an aqueous polyacrylamide solution,
b) injecting an aqueous fluid comprising the aqueous polyacrylamide solution into the underground mineral oil deposit through at least one injection well, and
c) withdrawing crude oil from the underground mineral oil deposit through at least one production well,
wherein a) comprises:
hydrating acrylonitrile in water in the presence of a biocatalyst, to obtain an acrylamide solution, wherein the biocatalyst is capable of converting acrylonitrile to acrylamide,
directly polymerizing the acrylamide solution, to obtain a polyacrylamide gel, wherein the polyacrylamide gel comprises 16 to 50% by weight of polyacrylamide solids, and
directly dissolving the polyacrylamide gel by adding water, to obtain an aqueous polyacrylamide solution,
wherein the aqueous polyacrylamide solution is prepared on an oil field.

40. A process for mining, mineral processing and/or metallurgy, the process comprising:
a) preparing an aqueous polyacrylamide solution, and
b) separating a solid and a liquid, disposing of tailings, depositing polymer modified tailings, managing tailings, modifying a density or a rheological property, aiding an agglomeration, binding, and/or handling a material with the aqueous polyacrylamide solution, wherein a) comprises:
hydrating acrylonitrile in water in the presence of a biocatalyst, to obtain an acrylamide solution, wherein the biocatalyst is capable of converting acrylonitrile to acrylamide,
directly polymerizing the acrylamide solution, to obtain a polyacrylamide gel, wherein the polyacrylamide gel comprises 16 to 50% by weight of polyacrylamide solids, and
directly dissolving the polyacrylamide gel by adding water, to obtain an aqueous polyacrylamide solution,
wherein the aqueous polyacrylamide solution is prepared in a mining area.


SEQUENCE LISTING (5 sequences)

SEQ ID NO: 1 - DNA, 612 bp, Rhodococcus rhodochrous
gtgagcgagcacgtcaataagtacacggagtacgaggcacgtaccaaggcgatcgaaaccttgctgtacgagcgagggctcatcacgcccgccgcggtcgaccgagtcgtttcgtactacgagaacgagatcggcccgatgggcggtgccaaggtcgtggccaagtcctgggtggaccctgagtaccgcaagtggctcgaagaggacgcgacggccgcgatggcgtcattgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggtttgggacagcagctccgaaatccgctacatcgtcatcccggaacggccggccggcaccgacggttggtccgaggaggagctgacgaagctggtgagccgggactcgatgatcggtgtcagtaatgcgctcacaccgcaggaagtgatcgtatga

SEQ ID NO: 2 - protein, 203 aa, Rhodococcus rhodochrous
VSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEEELTKLVSRDSMIGVSNALTPQEVIV

SEQ ID NO: 3 - DNA, 690 bp, Rhodococcus rhodochrous
atggatggtatccacgacacaggcggcatgaccggatacggaccggtcccctatcagaaggacgagcccttcttccactacgagtgggagggtcggaccctgtcaattctgacttggatgcatctcaagggcatatcgtggtgggacaagtcgcggttcttccgggagtcgatggggaacgaaaactacgtcaacgagattcgcaactcgtactacacccactggctgagtgcggcagaacgtatcctcgtcgccgacaagatcatcaccgaagaagagcgaaagcaccgtgtgcaagagatccttgagggtcggtacacggacaggaagccgtcgcggaagttcgatccggcccagatcgagaaggcgatcgaacggcttcacgagccccactccctagcgcttccaggagcggagccgagtttctctctcggtgacaagatcaaagtgaagagtatgaacccgctgggacacacacggtgcccgaaatatgtgcggaacaagatcggggaaatcgtcgcctaccacggctgccagatctatcccgagagcagctccgccggcctcggcgacgatcctcgcccgctctacacggtcgcgttttccgcccaggaactgtggggcgacgacggaaacgggaaagacgtagtgtgcgtcgatctctgggaaccgtacctgatctctgcgtga

SEQ ID NO: 4 - protein, 229 aa, Rhodococcus rhodochrous
MDGIHDTGGMTGYGPVPYQKDEPFFHYEWEGRTLSILTWMHLKGISWWDKSRFFRESMGNENYVNEIRNSYYTHWLSAAERILVADKIITEEERKHRVQEILEGRYTDRKPSRKFDPAQIEKAIERLHEPHSLALPGAEPSFSLGDKIKVKSMNPLGHTRCPKYVRNKIGEIVAYHGCQIYPESSSAGLGDDPRPLYTVAFSAQELWGDDGNGKDVVCVDLWEPYLISA

SEQ ID NO: 5 - protein, 203 aa, Rhodococcus rhodochrous
MSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEEELTKLVSRDSMIGVSNALTPQEVIV